(19) United States
(12) Patent Application Publication
(10) Pub. No.: US 2011/0319349 A1
(43) Pub. Date: Dec. 29, 2011

(54) Methods for Reducing Platelet Activation and for the Treatment of Thrombotic Events

(76) Inventors: Robert A.D. Scott, Roswell, GA (US); Victor Serebruany, West Friendship, MD (US)

(21) Appl. No.: 13/225,951

(22) Filed: Sep. 6, 2011

Related U.S. Application Data
(63) Continuation of application No. 11/544,845, filed on Oct. 6, 2006.
(60) Provisional application No. 60/724,109, filed on Oct. 6, 2005.

(51) Int. Cl. A61K 31/225; A61K 31/70; A61P 3/04; A61P 9/10; A61P 7/02; A61K 31/192; A61K 31/10

(52) U.S. Cl. 514/23; 514/571; 514/548; 514/712

(57) ABSTRACT

Methods and compositions for the treatment or prophylaxis of a disorder associated with platelet activation or enhanced thrombin activity are provided, that include the administration of an effective amount of a compound of the formula:

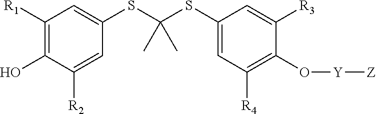

or its pharmaceutically acceptable salt, ester or prodrug, wherein the substituents are defined herein, optionally in the appropriate pharmaceutically acceptable carrier for the route of administration selected.

### CROSS REFERENCE TO RELATED APPLICATIONS

This application claims the benefit of priority to U.S. Provisional Application No. 60/724,109, filed Oct. 6, 2005, the disclosure of which is incorporated herein by reference.

### FIELD OF THE INVENTION

This invention provides methods for treating or preventing conditions associated with platelet activation and/or thrombin formation, using the disclosed compounds and compositions.

### BACKGROUND

Platelets are discoid cells found in large numbers in blood, which are important for blood coagulation and hemostasis. Upon activation by various stimuli like thrombin, thromboxane and ADP, platelets change into a spheroid shape with filopodia, degranulate and aggregate. Platelet activation is important for hemostasis and underlies various pathological conditions such as unstable angina pectoris, myocardial infarction, stroke, and coagulopathies. One of the physiological agents that activate platelets is thrombin, a serine protease. Thrombin mediates its action through the activation of protease activated receptors (PARs), including PAR-1 and PAR-4. See, e.g., U.S. Pat. No. 6,444,695.

While platelets are essential for normal blood clotting, overactive platelets can contribute to pathology. Platelet activation is the cause or a significant contributor to several vascular and non-vascular diseases. Platelet-dependent arterial thrombosis is known to trigger most heart attacks and strokes (Khan M L et al. Nature. Aug. 13, 1998; 394(6694):690-4).

The benefit of fibrinolytic therapy for the treatment of acute myocardial infarction has been demonstrated. (Lancet 1(8478):397-402, (1986); Lancet 2(8607):349-360, (1988); N. Engl. J. Med. 14(23):1465-1471, (1986); Lancet 1(8585):545-549, (1988); Wilcox, R G et al. (1988) Lancet 2(8610):525-530).

A variety of anti-clotting agents have been developed in the art, including anti-platelet agents and anti-coagulants. Anti-platelet drugs developed in the art include aspirin, which is the most common anti-clotting drug; glycoprotein IIb/IIIa inhibitors, such as abciximab (ReoPro®, Eli Lilly & Co.), eptifibatide (Integrilin®, Schering Plough Corp., Millenium Pharmaceuticals, and Glaxo Smith Kline), tirofoban (Aggrastat®, Merck & Co., Inc.) and lamifiban; and inhibitors of ADP-induced platelet activation, including thienopyridines, such as clopidogrel (Plavix®, Sanofi-Bristol Myers Squibb) and ticlopidine (Ticlid®, Roche Laboratories). Anti-coagulants developed in the art include heparin, such as standard unfractionated heparin, and low molecular weight heparins (LMWHs), such as ardeparin, dalteparin, enoxaparin and tinzaparin.

Ardeparin (Normiflo®; Wyeth-Ayerst Laboratories) is FDA-approved for the management, prophylaxis and/or treatment of deep venous thrombosis (DVT), a condition in which harmful blood clots form in the blood vessels of the legs. See U.S. Pat. No. 4,757,057 (Fussi et al). Dalteparin (Fragmin®; Pharmacia & Upjohn) is also FDA-approved for the treatment of DVT. See U.S. Pat. No. 4,303,651 (Lindhal et al.). Tinzaparin (Innohep®; Dupont) is a LMWH also used for the prevention and/or treatment of DVT. See Friedel H A et al., Drugs (1994) 48:638-60.

Enoxaparin (Lovenox®; Aventis Pharmaceuticals) is approved for multiple indications in the United States for the treatment of thromboembolic disease. See U.S. Pat. No. 4,692,435 (to Lormeau et al). U.S. Pat. No. 5,389,618 (to Debrie) discloses heterogeneous intimate admixtures of sulfated heparinic polysaccharides for the prophylaxis/treatment of acute thrombotic episodes.

Thrombin inhibitors known in the art include argatroban, danaproid and lepirudin. Lepirudin (Refludan®; Berlex Laboratories) ([Leu¹, Thr²]-63-desulfohirudin) is a thrombin inhibitor approved by the FDA (1998) for anticoagulation in patients with HIT (heparin-induced thrombocytopenia) and associated thromboembolic disease. It is a recombinant hirudin (see U.S. Pat. No. 5,180,668). Bivalirudin (Angiomax®; The Medicines Company) is a synthetic peptide that is a thrombin inhibitor approved as an anticoagulant in the U.S. for use in patients undergoing coronary angioplasty procedures. See U.S. Pat. No. 5,196,404. Argatroban (Glaxo Smith Kline) (5-[(aminoiminomethyl)amino]-1-oxo-2-[[(1,2,3,4-tetrahydro-3-methyl-8-quinolinyl)-sulfonyl]amino]pentyl]-4-methyl-2-piperidinecarboxylic acid, monohydrate) is a thrombin inhibitor approved as an anticoagulant in patients with or at risk for HIT undergoing percutaneous coronary intervention. See U.S. Pat. No. 5,214,052.

There remains a need for new anti-platelet agents and thrombin inhibitors.

U.S. Pat. No. 5,262,439 to Parthasarathy, which is assigned to AtheroGenics, Inc. discloses analogs of probucol with increased water solubility in which one or both of the hydroxyl groups are replaced with ester groups that increase the water solubility of the compound. In one embodiment, the derivative is selected from the group consisting of a mono- or di-probucol ester of succinic acid, glutaric acid, adipic acid, seberic acid, sebacic acid, azelaic acid, or maleic acid. In another embodiment, the probucol derivative is a mono- or di-ester in which the ester contains an alkyl or alkenyl group that contains a functionality selected from the group consisting of a carboxylic acid group, amine group, salt of an amine group, amide groups, salt of an amide groups, and aldehyde groups.

A series of French patents disclose that certain probucol derivatives are hypocholesterolemic and hypolipemic agents: Fr 2168137 (bis 4hydroxyphenylthioalkane esters); Fr 2140771 (tetralinyl phenoxy alkanoic esters of probucol); Fr 2140769 (benzofuryloxyalkanoic acid derivatives of probucol); Fr 2134810 (bis-(3-alkyl-5-t-alkyl-4-thiazole-5-carboxy)phenylthio)alkanes; FR 2133024 (bis-(4 nicotinoyloxyphenylthio)-propanes; and Fr 2130975 (bis(4-phenoxyalkanoyloxy)phenylthio)alkanes).

U.S. Pat. No. 5,155,250 to Parker, et al. discloses that 2,6-dialkyl-4-silylphenols are antiatherosclerotic agents. The same compounds are disclosed as serum cholesterol lowering agents in PCT Publication No. WO 95/15760, published on Jun. 15, 1995. U.S. Pat. No. 5,608,095 to Parker, et al. discloses that alkylated-4-silyl-phenols inhibit the peroxidation of LDL, lower plasma cholesterol, and inhibit the expression of VCAM-1, and thus are useful in the treatment of atherosclerosis.

A series of European patent applications and to Shionogi Seiyaku Kabushiki Kaisha disclose phenol thioethers for use in treating arteriosclerosis. European Patent Application No. 348 203 discloses phenolic thioethers which inhibit the denaturation of LDL and the incorporation of LDL by macrophages. The compounds are useful as anti-arteriosclerosis agents. Hydroxamic acid derivatives of these compounds are disclosed in European Patent Application No. 405 788 and are useful for the treatment of arteriosclerosis, ulcer, inflammation and allergy. Carbamoyl and cyano derivatives of the phenolic thioethers are disclosed in U.S. Pat. No. 4,954,514 to Kita, et al.

U.S. Pat. No. 6,121,319, which issued on Sep. 19, 2000, and corresponding WO 98/51662 filed by AtheroGenics, Inc. and published on Nov. 18, 1998, describes certain probucol derivatives for the treatment of disorders mediated by vascular cell adhesion molecule-1 (VCAM-1), and inflammatory and cardiovascular disorders. WO 01/70757 filed by AtheroGenics, Inc. and published on Sep. 27, 2001 describes thioketals and thioethers for the inhibition of VCAM-1. U.S. Pat. No. 6,147,250, filed by AtheroGenics, Inc. on May 14, 1998, discloses compounds, compositions and methods for inhibiting the expression of VCAM-1.

Meng et al., discloses a series of phenolic inhibitors of TNF-α-inducible expression of VCAM-1 with concurrent antioxidant and lipid-modulating properties. The compounds disclosed have demonstrated efficacies in animal models of atherosclerosis and hyperlipidemia. (Novel Phenolic Antioxidants As Multifunctional Inhibitors Of Inducible VCAM-1 Expression For Use In Atherosclerosis, Bioorganic & Medl Chem Ltrs. 12(18), 2545-2548, 2002).

Sundell et al., discloses a metabolically stable phenolic antioxidant compound derived from probucol. ([4-[[1-[[3,5-bis(1,1-dimethylethyl)-4-hydroxypehenyl]thio]-1-methylethyl]thio]2,6-bis(1,1-dimethylethyl)phenoxy]acetic acid) inhibits TNF-α-stimulated endothelial expression of VCAM-1 and MCP-1, two redox-sensitive inflammatory genes critical for the recruitment of leukocytes to joints in rheumatoid arthritis (RA), to a greater extent than ICAM-1. (AGIX-4207: A Novel Antioxidant And Anti-Inflammatory Compound Inhibits Progression Of Collagen II Arthritis In The Rat, FASEB Journal Vol. 16, November 4, PP. A182, Mar. 20, 2002. Apr. 20-24, 2002, Annual Meeting of the Professional Research Scientists on Experimental Biology, ISSN 0892-6638).

There remains a need for treatments for patients suffering from a vascular event, disease or disorder associated with the activation of platelets and/or elevated thrombin levels, such as acute myocardial infarction and stroke.

### SUMMARY

It has been discovered that certain phenolic antioxidants, or their pharmaceutically acceptable salts, esters or prodrugs, are useful for treating or preventing conditions that are associated with platelet activation or elevated thrombin levels. The phenolic antioxidants described herein surprisingly have antiplatelet activity that allows them to be used in a variety of therapeutic applications to prevent or treat thrombotic events, such as stroke, angina and pulmonary embolism.

In a particular embodiment, a method for treating or preventing a vascular condition in an individual is provided, wherein the vascular condition is associated with platelet activation, elevated thrombin levels or elevated thrombin receptor activity or expression, and wherein the method comprises administering an effective amount of a phenolic antioxidant as disclosed herein, or pharmaceutically acceptable salt or ester thereof, optionally in a pharmaceutically acceptable carrier.

The phenolic antioxidant compounds disclosed herein can be used in methods for treating a vascular event, disease or disorder that is present in an individual, or which the individual is at risk of being afflicted with. In a particular embodiment, the vascular condition is a thrombotic or thromboembolic event. Thrombotic events and disorders include, for example, acute myocardial infarction, unstable angina, ischemic stroke, pulmonary embolism, transient ischemic attack and deep vein thrombosis. The method may include treating humans or animals.

Compounds useful in the methods and compositions disclosed herein include those listed in the section herein entitled “Compounds”.

The compound may be administered by any suitable method including, for example, orally, intravenously, intramuscularly, subcutaneously, parenterally, nasally, by inhalation, by implant, or by suppository. In one embodiment, the compound is administered orally in an amount between about 0.5 mg-2500 mg/daily.

Optionally the method may further comprise administering a second therapeutic agent such as a anti-platelet drug or an anticoagulant. Optionally, the compound is provided with or administered sequentially in combination with (i) an anticoagulant such as unfractionated heparin, heparin or hirudin; (ii) a thrombolytic agent such as streptokinase, alteplase, reteplase, monteplase, lanoteplase, saruplase, urokinase, pro-urokinase, staphylokinase, tenecteplase, or anisoylated plasminogen-streptokinase activator complex; or (iii) an anti-platelet drug such as abciximab, eptifibatide, tirofoban, lamifiban, aspirin, ticlopidine, clopidogrel, dipyridamole, or Aggrenox®.

In one embodiment, the compound is administered between about 6 to 24 hours, about 12 to 24 hours, about 18 to 24 hours, or about 20 to 24 hours after a thrombolysis has occurred. In one embodiment, the compound is administered multiple times.

In a further embodiment, a method for reducing a platelet activation state of an individual in need thereof is provided, the method comprising administering an effective amount of a phenolic antioxidant compound.

Optionally, the method includes comparing the level of platelet activation from a sample taken from the individual after administration of the compound to a control, and detecting a decrease in platelet activation. In a particular embodiment, a method is provided for comparing the level of at least one platelet activation marker (PAM) from a sample taken from an individual, to a control, and detecting a decrease after administration of the phenolic antioxidant compound. Platelet activation markers include, for example, CD9, GPIb, GPIIb, CDIa-IIa, P-selectin, PECAM-1, vitronectin, integrins and adhesive molecules. Optionally, the level of the PAM is reduced by at least about 10%, 15%, 17%, or 20% or more.

In a still further embodiment, provided is a method of reducing or inhibiting platelet aggregation in an individual in need thereof, the method comprising administering an effective amount of a phenolic antioxidant compound. In a particular embodiment, the method includes reducing platelet aggregation by at least about 10%.

Optionally, the method includes comparing the level of platelet aggregation from a sample taken from the individual after administration of the compound to a control, and detecting a decrease in platelet aggregation.

In another embodiment, a method for reducing thrombin levels, or inhibiting thrombin formation in an individual in need thereof is provided, the method comprising administering an effective amount of a phenolic antioxidant compound as disclosed herein.

Optionally, the method includes comparing the level of thrombin from a sample taken from the individual after administration of the compound to a control, and detecting a decrease in thrombin.

In a further embodiment, provided is a method for reducing the activity or expression of protease activating receptors (PAR), including PAR-1 and PAR-4 in platelets of an individual, by administering an effective amount of a phenolic antioxidant compound.

Optionally, the method includes comparing the activity or expression level of platelet PAR-1/PAR-4 thrombin receptor expression from a sample taken from the individual after administration of the compound to a control, and detecting a decrease in activity or expression.

In a particular embodiment, the compound used in the methods described herein is a compound of the formula

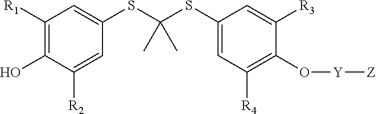

or a pharmaceutically acceptable salt thereof wherein:
Y is a bond or

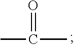

R₁, R₂, R₃, and R₄ are independently selected from the group consisting of hydrogen, hydroxy, alkoxy, C₁₋₁₀alkyl, aryl, heteroaryl, C₁₋₁₀alkaryl, and aryl C₁₋₁₀alkyl, wherein said alkoxy, C₁₋₁₀alkyl, aryl, heteroaryl, C₁₋₁₀alkaryl, and aryl C₁₋₁₀alkyl may optionally be substituted with one or more moiety selected from C₁₋₁₀alkyl, halogen, nitro, amino, haloC₁₋₁₀alkyl, C₁₋₁₀alkylamino, diC₁₋₁₀alkylamino, acyl, and acyloxy;
Z is selected from the group consisting of C₁₋₁₀alkyl, C₂₋₁₀alkenyl, C₂₋₁₀alkynyl, hydroxyC₁₋₁₀alkyl, aryl, heteroaryl, C₁₋₁₀alkaryl, arylC₁₋₁₀alkyl, heteroarylC₁₋₁₀alkyl, C₁₋₁₀alkoxyC₁₋₁₀alkyl, C₁₋₁₀alkylaminoC₁₋₁₀alkyl, carboxyC₁₋₁₀alkyl, C₁₋₁₀dialkylaminoC₁₋₁₀alkyl, aminoC₁₋₁₀alkyl, heterocycle, R₇NH, R₇R₇N, and carboxy, wherein any may optionally be substituted by one or more R₅;
R₅ is independently selected from the group selected from hydroxy, C₁₋₁₀alkyl, C₁₋₁₀alkoxy, C₂₋₁₀alkenyl, heterocycle, halo, nitro, amino, cyano, C₁₋₁₀alkylamino, diC₁₋₁₀alkylamino, acyl, acyloxy, COOH, COOR₇, OC(O)R₇, CH(OH)R₇, NHR₇, NR₇R₇, C(O)NH₂, C(O)NHR₇, CONR₇R₇, NHC(O)O—R₇, OSO₃H, SO₃H, SO₂NHR₇, SO₂NR₇R₇, P(O)(OH)OR₇, PO₂H₂ P(O)(OH)R₇, P(O)(OR₇)₂, P(O)R₇(OR₇), OPO₃H, PO₃H₂, hydroxymethyl, and cyclic phosphate, wherein when possible, all may be optionally substituted by one or more R₆;
R₆ is independently selected from the group consisting of hydroxy, C₁₋₁₀alkyl, C₁₋₁₀alkoxy, acyloxy, halo, nitro, amino, cyano, C₁₋₁₀alkylamino, diC₁₋₁₀alkylamino, acyl, and acyloxy;
R₇ is independently selected from the group consisting of C₁₋₁₀alkyl, C₂₋₁₀alkenyl, C₂₋₁₀alkynyl, C₁₋₁₀alkoxy, C₁₋₁₀alkoxycarbonylC₁₋₁₀alkyl, aryl, carboxyC₁₋₁₀alkyl, C₁₋₁₀alkylcarboxyC₁₋₁₀alkyl, C₁₋₁₀alkylcarboxyC₁₋₁₀aryl, heterocycle, hetercycleC₁₋₁₀alkyl, and heteroaryl, wherein any may be optionally substituted by one or more R₈; and
R₈ is independently selected from the group consisting of hydroxy, C₁₋₁₀alkyl, C₁₋₁₀alkoxy, acyloxy, halo, nitro, amino, cyano, and carboxy;
wherein two R₇ groups may come together to form a 4 to 7 membered ring.

In a particular embodiment the compound is:

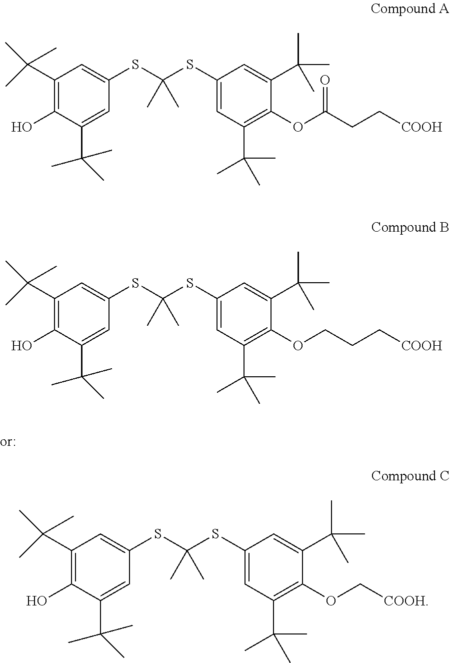

### DETAILED DESCRIPTION

It has been discovered that certain phenolic antioxidant compounds, or their pharmaceutically acceptable salts or prodrugs, are useful for reducing or inhibiting platelet activation or aggregation, or reducing thrombin levels, or reducing the activity or expression of thrombin receptors. The invention therefore provides a new approach for the treatment and/or prevention of thrombotic or thromboembolic disorders accompanied or characterized by platelet activation, elevated thrombin levels or increased expression of thrombin receptors.

### Definitions

The term alkyl, as used herein, unless otherwise specified, refers to a saturated straight, branched, or cyclic, primary, secondary, or tertiary hydrocarbon of for example C₁ to C₁₀, and specifically includes methyl, ethyl, propyl, isopropyl, cyclopropyl, butyl, isobutyl, t-butyl, pentyl, cyclopentyl, isopentyl, neopentyl, hexyl, isohexyl, cyclohexyl, cyclohexylmethyl, 3-methylpentyl, 2,2-dimethylbutyl, and 2,3-dimethylbutyl, trifluoromethyl and perfluoroalkyl. The alkyl is optionally substituted. The alkyl group can be substituted with any moiety that does not adversely affect the properties of the active compound, for example, but not limited to hydroxyl, halo (including independently F, Cl, Br, and I), perfluoro alkyl including trifluoromethyl, amino, alkylamino, arylamino, alkoxy, aryloxy, nitro, cyano, acyl, amido, carboxamido, carboxylate, thiol, alkylthio, azido, sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991, hereby incorporated by reference. In one embodiment, the alkyl can be, for example, CF₃, CH₂CF₃, CCl₃, or cyclopropyl.

In the text, whenever any range is used, the range independently and separately includes every member of the range. As a nonlimiting example, the range of C₁-C₁₀ includes independently C₁, C₂, C₃, C₄, C₅, C₆, C₇, C₈, C₉, and C₁₀.

In the text, whenever the term C(alkyl range) is used, the term independently includes each member of that class as if specifically and separately set out. As a nonlimiting example, the term “C₁₋₁₀” independently represents each species that falls within the scope, including, but not limited to, methyl, ethyl, propyl, isopropyl, butyl, sec-butyl, iso-butyl, tert-butyl, pentyl, iso-pentyl, neo-pentyl, cyclopentyl, cyclopentyl, hexyl, 1-methylpentyl, 2-methylpentyl, 3-methylpentyl, 4-methylpentyl, 1-ethylbutyl, 2-ethylbutyl, 3-ethylbutyl, 4-ethyl butyl, cyclohexyl, heptyl, 1-methylhexyl, 2-methylhexyl, 3-methylhexyl, 4-methylhexyl, 5-methylhexyl, 6-methylhexyl, 1-ethylpentyl, 2-ethylpentyl, 3-ethylpentyl, 4-ethylpentyl, 5-ethylpenyl, 1-propylbutyl, 2-propylbutyl, 3-propybutyl, 4-propylbutyl, cycloheptyl, octyl, 1-methylheptyl, 2-methylheptyl, 3-methylheptyl, 4-methylheptyl, 5-methylheptyl, 6-methylheptyl, 7-methylheptyl, 1-ethylhexyl, 2-ethylhexyl, 3-ethylhexyl, 4-ethylhexyl, 5-ethylhexyl, 6-ethylhextyl, 1-propylpentyl, 2-propylpentyl, 3-propypentyl, 4-propylpentyl, 5-propylpentyl, cyclooctyl, nonyl, cyclononyl, decyl, or cyclodecyl. C₂₋₁₀ and C₁₋₆ likewise can independently include any of its member groups, as if each were independently named herein.

The term “alkylene” radical denotes a divalent alkane such as a linear or branched radical including those having from 2 to 10 carbon atoms or 2 to 6 carbon atoms and having attachment points for two or more covalent bonds. Examples of such radicals are methylene, ethylene, methylethylene, and isopropylidene. Included within the scope of this term are 1,2-ethane-diyl, 1,1-ethane-diyl, 1,3-propane-diyl, 1,2-propane-diyl, 1,3-butane-diyl, 1,4-butane-diyl and the like. The alkylene group or other divalent moiety disclosed herein can be optionally substituted with one or more moieties selected from the group consisting of alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term “alkenyl” refers to an unsaturated, acyclic hydrocarbon radical, linear or branched, in so much as it contains one or more double bonds, including such radicals containing about 2 to 10 carbon atoms or having from 2 to 6 carbon atoms. The alkenyl group may be optionally substituted in the same manner as described for alkyl groups. Examples of suitable alkenyl radicals include ethenyl, propenyl, hydroxypropenyl, buten-1-yl, buten-2-yl, penten-1-yl, penten-2-yl, 4-methoxypenten-2-yl, 3-methylbuten-1-yl, hexen-1-yl, hexen-2-yl, hexen-3-yl, 3,3-dimethylbuten-1-yl radicals and the like.

The term “alkynyl” refers to an unsaturated, acyclic hydrocarbon radical, linear or branched, in so much as it contains one or more triple bonds, including such radicals containing about 2 to 10 carbon atoms or having from 2 to 6 carbon atoms. The alkynyl group may be optionally substituted in the same manner as described for alkyl groups. Examples of suitable alkynyl radicals include ethynyl, propynyl, hydroxypropynyl, butyn-1-yl, butyn-2-yl, pentyn-1-yl, pentyn-2-yl, 4-methoxypentyn-2-yl, 3-methylbutyn-1-yl, hexyn-1-yl, hexyn-2-yl, hexyn-3-yl, 3,3-dimethylbutyn-1-yl radicals and the like.

The term “acyl”, alone or in combination, means a carbonyl or thionocarbonyl group bonded to a radical selected from, for example, hydrido, alkyl, alkenyl, alkynyl, haloalkyl, alkoxy, alkoxyalkyl, haloalkoxy, aryl, heterocyclyl, heteroaryl, alkylsulfinylalkyl, alkylsulfonylalkyl, aralkyl, cycloalkyl, cycloalkylalkyl, cycloalkenyl, alkylthio, arylthio, amino, alkylamino, dialkylamino, aralkoxy, arylthio, and alkylthioalkyl. Examples of “acyl” are formyl, acetyl, benzoyl, trifluoroacetyl, phthaloyl, malonyl, nicotinyl, and the like.

The terms “alkoxy” and “alkoxyalkyl” embrace linear or branched oxy-containing radicals each having alkyl portions of one to about ten carbon atoms, such as methoxy radical. The term “alkoxyalkyl” also embraces alkyl radicals having one or more alkoxy radicals attached to the alkyl radical, that is, to form monoalkoxyalkyl and dialkoxyalkyl radicals. Other alkoxy radicals are “lower alkoxy” radicals having one to six carbon atoms. Examples of such radicals include methoxy, ethoxy, propoxy, butoxy and tert-butoxy alkyls. The “alkoxy” radicals may be further substituted with one or more halo atoms, such as fluoro, chloro or bromo, to provide “haloalkoxy” radicals. Examples of such radicals include fluoromethoxy, chloromethoxy, trifluoromethoxy, difluoromethoxy, trifluoroethoxy, fluoroethoxy, tetrafluoroethoxy, pentafluoroethoxy, and fluoropropoxy.

The term “acyloxy” denotes oxy-containing radicals each having an acyl portion.

The term “alkylamino” denotes “monoalkylamino” and “dialkylamino” containing one or two alkyl radicals, respectively, attached to an amino radical. The terms arylamino denotes “monoarylamino” and “diarylamino” containing one or two aryl radicals, respectively, attached to an amino radical. The term “aralkylamino”, embraces aralkyl radicals attached to an amino radical. The term aralkylamino denotes “monoaralkylamino” and “diaralkylamino” containing one or two aralkyl radicals, respectively, attached to an amino radical. The term aralkylamino further denotes “monoaralkyl monoalkylamino” containing one aralkyl radical and one alkyl radical attached to an amino radical.

The term “aryl”, alone or in combination, means a carbocyclic aromatic system containing one, two or three rings wherein such rings may be attached together in a pendent manner or may be fused. The term “aryl” embraces aromatic radicals such as phenyl, naphthyl, tetrahydronaphthyl, indane and biphenyl. Said “aryl” group may have 1 to 5 substituents termed “heteroaryl” such as heteroarylamino, N-aryl-N-alkylamino, N-heteroarylamino-N-alkylamino, haloalkylthio, alkanoyloxy, alkoxy, heteroaralkoxy, cycloalkoxy, cycloalkenyloxy, hydroxy, amino, thio, nitro, lower alkylamino, alkylthio, alkylthioalkyl, arylamino, aralkylamino, arylthio, alkylsulfinyl, alkylsulfonyl, alkylsulfonamido, alkylaminosulfonyl, amidosulfonyl, monoalkyl amidosulfonyl, dialkyl amidosulfonyl, monoarylamidosulfonyl, arylsulfonamido, diarylamidosulfonyl, monoalkyl monoaryl amidosulfonyl, arylsulfinyl, arylsulfonyl, heteroarylthio, heteroarylsulfinyl, heteroarylsulfonyl, alkanoyl, alkenoyl, aroyl, heteroaroyl, aralkanoyl, heteroaralkanoyl, haloalkanoyl, alkyl, alkenyl, alkynyl, alkylenedioxy, haloalkylenedioxy, cycloalkyl, cycloalkenyl, lower cycloalkylalkyl, lower cycloalkenylalkyl, halo, haloalkyl, haloalkoxy, hydroxyhaloalkyl, hydroxyaralkyl, hydroxyalkyl, hydoxyheteroaralkyl, haloalkoxyalkyl, aryl, aralkyl, aryloxy, aralkoxy, aryloxyalkyl, saturated heterocyclyl, partially saturated heterocyclyl, heteroaryl, heteroaryloxy, heteroaryloxyalkyl, arylalkyl, heteroarylalkyl, arylalkenyl, heteroarylalkenyl, carboalkoxy, carboaralkoxy, cyano, and carbohaloalkoxy.

The term “heteroaryl or heteroaromatic base,” as used herein, refers to an aromatic that includes at least one sulfur, oxygen, nitrogen or phosphorus in the aromatic ring. The term “heterocyclic base” refers to a nonaromatic cyclic group wherein there is at least one heteroatom, such as oxygen, sulfur, nitrogen or phosphorus in the ring. Nonlimiting examples of heteroaryl and heterocyclic groups include pyrimidines, such as thymine, cytosine and uracil, substituted pyrimidines such as N5-halopyrimidines, N5-alkylpyrimidines, N5-benzylpyrimidines, N5-vinylpyrimidine, N5-acetylenic pyrimidine, N5-acyl pyrimidine, 6-azapyrimidine, 2-mercaptopyrmidine, and in particular, 5-fluorocytidinyl, 5-azacytidinyl, 5-azauracilyl, purines such as adenine, guanine, inosine and pteridine, substituted purines such as N6-alkylpurines, N6-benzylpurine, N6-halopurine, N6-vinypurine, N6-acetylenic purine, N6-acyl purine, N6-thioalkyl purine, N6-hydroxyalkyl purine, N6-thioalkyl purine and N5-hydroxyalkyl purine and in particular, 6-chloroadenine and 6-azoadenine, triazolopyridinyl, imidazolopyridinyl, pyrrolopyrimidinyl, pyrazolopyrimidinyl, pyridine, pyrrole, indole, imidazole, pyrazole, quinazoline, pyridazine, pyrazine, cinnoline, phthalazine, quinoxaline, xanthine, hypoxanthine, triazolopyridine, imidazolepyridine, imidazolotriazine, pyrrolopyrimidine, pyrazolopyrimidine, 1-triphenyl-methyltetrazolyl, 2-triphenylmethyl-tetrazolyl group, furyl, furanyl, thienyl, isothiazolyl, imidazolyl, tetrazolyl, pyrazinyl, benzofuranyl, benzothiophenyl, quinolyl, isoquinolyl, benzothienyl, isobenzofuryl, pyrazolyl, indolyl, isoindolyl, benzimidazolyl, purinyl, carbazolyl, oxazolyl, thiazolyl, isothiazolyl, 1,2,4-thiadiazolyl, isooxazolyl, pyrrolyl, quinazolinyl, cinnolinyl, phthalazinyl, xanthinyl, hypoxanthinyl, thiophene, furan, pyrrole, isopyrrole, pyrazole, imidazole, 1,2,3-triazole, oxazole, thiazole, isothiazole, pyridazine, and pteridinyl, aziridines, thiazole, 1,2,3-oxadiazole, thiazine, pyridine, pyrazine, piperazine, pyrrolidine, oxaziranes, phenazine, phenothiazine, morpholinyl, pyrazolyl, pyridazinyl, pyrazinyl, quinoxalinyl, xanthinyl, hypoxanthinyl, pteridinyl, isoxazolyl, pyrrolidin-2-yl, piperidin-2-yl, quinolin-2-yl, isoquinolin-1-yl, pyridin-2-yl, 4-methylimidazol-2-yl, 1-methylimidazol-4-yl, 1-n-hexylimidazol-4-yl, 1-benzylimidazol-4-yl, 1,2-dimethylimidazol-4-yl, 1-n-pentyl-2-methyl-imidazol-4-yl, 1-benzyl-2-methyl-imidazol-5-yl, benzimidazol-2-yl, 1-methylbenzimidazol-2-yl, 1-methyl-5-methoxy-benzimidazol-2-yl, imidazo[1,2-a]pyridin-2-yl, 6-chloro-imidazo[1,2-a]-pyridin-2-yl, imidazo[1,2-a]pyrimidin-2-yl, 2-phenyl-imidazo[2,1-b]-thiazol-6-yl, purin-8-yl, imidazo[4,5-b]pyrazin-2-yl, 5-methyl-imidazolidin-2,4-dion-3-yl, 2-n-propyl-pyridazin-3-on-6-yl, oxazol-4-yl, 2-isopropyl-thiazol-4-yl, 1-ethyl-imidazol-4-yl, 1-(4-fluorobenzyl)-2-methyl-imidazol-4-yl, 1-aminocarbonylmethyl-imidazol-4-yl, 1-morpholino-carbonylmethyl-imidazol-4-yl, 2-isopropyl-pyridazin-3-on-6-yl, 2-benzyl-pyridazin-3-on-6-yl, 2-(2-phenylethyl)-pyridazin-3-on-6-yl, 2-(3-phenylpropyl)-pyridazin-3-on-6-yl, 4-methyl-pyridazin-3-on-6-yl, 5-methyl-pyridazin-3-on-6-yl, 4,5-dimethyl-pyridazin-3-on-6-yl, 2,4-dimethyl-pyridazin-3-on-6-yl, 2,5-dimethyl-pyridazin-3-on-6-yl, 2,4,5 -trimethyl-pyridazin-3-on-6-yl. The heteroaromatic or heterocyclic group can be optionally substituted with any desired moiety, including one or more moieties selected from the group consisting of alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference. The heteroaromatic can be partially or totally hydrogenated as desired. As a nonlimiting example, dihydropyridine can be used in place of pyridine. Functional oxygen and nitrogen groups on the heteroaryl group can be protected as necessary or desired. Suitable protecting groups are well known to those skilled in the art, and include trimethylsilyl, dimethylhexylsilyl, t-butyldimethylsilyl, and t-butyldiphenylsilyl, trityl or substituted trityl, alkyl groups, acyl groups such as acetyl and propionyl, methanesulfonyl, and p-toluenesulfonyl.

The term “alditol” as referred to herein, and unless otherwise specified, refers to a carbohydrate in which the aldehyde or ketone group has been reduced to an alcohol moiety. The alditols can also be optionally substituted or deoxygenated at one or more positions. Exemplary substituents include hydrogen, halo, haloalkyl, carboxyl, acyl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, thioester, thioether, oxime, hydrazine, carbamate, phosphonic acid, phosphonate, or any other viable functional group that does not inhibit the pharmacological activity of this compound. Particular exemplary substituents include amine and halo, particularly fluorine. The substituent or alditol can be either unprotected, or protected as necessary, as known to those skilled in the art, for example, as taught in Greene, et al., Protective Groups in Organic Synthesis, John Wiley and Sons, Second Edition, 1991, hereby incorporated by reference. The alditol may comprise 3, 4, 5, 6, or 7 carbons. Examples of useful alditols are those derived from reduction of monosaccharides, including specifically those derived from the reduction of pyranose and furanose sugars.

The term “carbohydrate” as referred to herein, and unless otherwise specified, refers to a compound of carbon, hydrogen, and oxygen that contains an aldehyde or ketone group in combination with at least two hydroxyl groups. The term “carbohydrate lactone” represents a carbohydrate, wherein the anomeric hydroxy group has been formally oxidized to a carbonyl group thus forming a substituted or unsubstituted cyclic ester or lactone. The carbohydrates and carbohydrate lactones can also be optionally substituted or deoxygenated at one or more positions. Carbohydrates and carbohydrate lactones thus include substituted and unsubstituted monosaccharides, disaccharides, oligosaccharides, and polysaccharides. The saccharide can be an aldose or ketose, and may comprise 3, 4, 5, 6, or 7 carbons. In one embodiment they are monosaccharides. In another embodiment they can be pyranose and furanose sugars. They can be optionally deoxygenated at any corresponding C-position, and/or substituted with one or more moieties such as hydrogen, halo, haloalkyl, carboxyl, acyl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, thioester, thioether, oxime, hydrazine, carbamate, phosphonic acid, phosphonate, or any other viable functional group that does not inhibit the pharmacological activity of this compound. Particular exemplary substituents include amine and halo, particularly fluorine. The substituent, carbohydrate, or carbohydrate lactone can be either unprotected, or protected as necessary, as known to those skilled in the art, for example, as taught in Greene, et al., Protective Groups in Organic Synthesis, John Wiley and Sons, Second Edition, 1991, hereby incorporated by reference.

The term “carboxyalkyl” denotes a carboxy group attached to an alkyl group.

The term “alkoxycarbonyl” denotes a radical having the formula alkyl-O—C(O)—, wherein alkyl is defined herein.

The term “cyano” radical denotes a carbon radical having three of four covalent bonds shared by a nitrogen atom.

The term “carbonyl” or “—C(O)—” denotes a carbon radical having two of the four covalent bonds shared with an oxygen atom. The term “carboxy” embraces a hydroxyl radical, attached to one of two unshared bonds in a carbonyl group. The term “alkoxy carbonyl” denotes a carbon radical having two of the four covalent bonds shared with an oxygen atom, and a third covalent bond shared with another oxygen, also denoted by

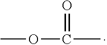

The term “halo” and “halogen” means halogens such as fluorine, chlorine, bromine or iodine atoms.

The term “hydroxyalkyl” embraces radicals wherein any one or more of the alkyl carbon atoms is substituted with a hydroxyl. Specifically embraced are monohydroxyalkyl, dihydroxyalkyl and polyhydroxyalkyl radicals.

The term “aminoalkyl” denotes an amino group attached to an alkyl group, for example -alkyl-NH₂.

The term “independently” is used herein to indicate that the variable which is independently applied varies independently from application to application. Thus, in a compound such as R″XYR″, wherein R″ is “independently carbon or nitrogen,” both R″ can be carbon, both R″ can be nitrogen, or one R″ can be carbon and the other R″ nitrogen.

The term “therapeutically effective amount” shall mean that amount of drug or pharmaceutical agent that will elicit the biological or medical response of a tissue, system, animal or human that is being sought.

The term “pharmaceutically acceptable salts” refer to salts or complexes that retain the desired biological activity of the compounds and exhibit minimal undesired toxicological effects. Nonlimiting examples of such salts are (a) acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalcturonic acid; (b) base addition salts formed with metal cations such as zinc, lithium, calcium, bismuth, barium, magnesium, aluminum, copper, cobalt, nickel, cadmium, sodium, potassium, and the like, or with a cation formed from ammonia, N,N-dibenzylethylenediamine, D-glucosamine, tetraethylammonium, or ethylenediamine; or (c) combinations of (a) and (b); e.g., a zinc tannate salt or the like. Also included in this definition are pharmaceutically acceptable quaternary salts known by those skilled in the art, which specifically include the quaternary ammonium salt of the formula —NR⁺A⁻, wherein R is e.g. trialkyl and A is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

In cases where compounds are sufficiently basic or acidic to form stable non-toxic acid or base salts, administration of the compounds as salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The term “pharmaceutically acceptable ester” as used herein, unless otherwise specified, includes those estrs of one or more compounds, which are, within the scope of sound medical judgment, suitable for use in contact with the tissues of hosts without undue toxicity, irritation, allergic response, and the like, are commensurate with a reasonable benfit/risk ratio, and are effective for their intended use.

The term “pharmaceuticaly acceptable prodrug” includes a compound that is metabolized, for example, hydrolyzed or oxidized, in the host to form an active compound. Typical examples of prodrugs include compounds that have biologically labile protecting groups on a functional moiety of the active compound. Prodrugs include ocmpounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound.

### Compounds

A variety of phenolic antioxidant compounds may be used in the methods and compositions described herein, as exemplified by the compounds described herein. Such compounds are sometimes referred to herein as “probucol monoester derivatives”.

In one embodiment, the phenolic antioxidant compound is described by the following formula:

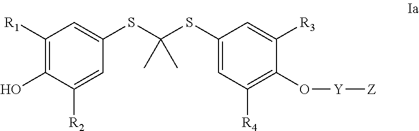

or a pharmaceutically acceptable salt thereof wherein:
Y is a bond or

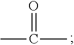

R₁, R₂, R₃, and R₄ are independently selected from the group consisting of hydrogen, hydroxy, alkoxy, C₁₋₁₀alkyl, aryl, heteroaryl, C₁₋₁₀alkaryl, and aryl C₁₋₁₀alkyl, wherein said alkoxy, C₁₋₁₀alkyl, aryl, heteroaryl, C₁₋₁₀alkaryl, and aryl C₁₋₁₀alkyl may optionally be substituted with one or more moiety from the group selected from C₁₋₁₀alkyl, halogen, nitro, amino, haloC₁₋₁₀alkyl, C₁₋₁₀alkylamino, diC₁₋₁₀alkylamino, acyl, and acyloxy;
Z is selected from the group consisting of C₁₋₁₀alkyl, C₂₋₁₀alkenyl, C₂₋₁₀alkynyl, hydroxyC₁₋₁₀alkyl, aryl, heteroaryl, C₁₋₁₀alkaryl, arylC₁₋₁₀alkyl, heteroarylC₁₋₁₀alkyl, C₁₋₁₀alkoxyC₁₋₁₀alkyl, C₁₋₁₀alkylaminoC₁₋₁₀alkyl, carboxyC₁₋₁₀alkyl, C₁₋₁₀dialkylaminoC₁₋₁₀alkyl, aminoC₁₋₁₀alkyl, heterocycle, R₇NH, R₇R₇N, carboxyC₁₋₁₀alkyl and carboxy, wherein any may optionally be substituted by one or more R₅;
R₅ is independently selected from the group selected from hydroxy, C₁₋₁₀alkyl, C₁₋₁₀alkoxy, halo, nitro, amino, cyano, C₁₋₁₀alkylamino, diC₁₋₁₀alkylamino, acyl, acyloxy, C₂₋₁₀alkenyl, C₂₋₁₀alkynyl, hydroxyC₁₋₁₀alkyl, aryl, heteroaryl, heterocycle, C₁₋₁₀alkaryl, arylC₁₋₁₀alkyl, heteroarylC₁₋₁₀alkyl, C₁₋₁₀alkoxyC₁₋₁₀alkyl, C₁₋₁₀alkylaminoC₁₋₁₀alkyl, carboxyC₁₋₁₀alkyl, C₁₋₁₀dialkylaminoC₁₋₁₀alkyl, aminoC₁₋₁₀alkyl, heterocycle, R₇NH, R₇R₇N, carboxyC₁₋₁₀alkyl, COOH, COOR₇, OC(O)R₇, CH(OH)R₇, NHR₇, NR₇R₇, C(O)NH₂, C(O)NHR₇, CONR₇R₇, NHC(O)O—R₇, OSO₃H, SO₃H, SO₂NHR₇, SO₂NR₇R₇, P(O)(OH)OR₇, PO₂H₂ P(O)(OH)R₇, P(O)(OR₇)₂, P(O)R₇(OR₇), OPO₃H, PO₃H₂, hydroxymethyl, and cyclic phosphate, wherein when possible, all may be optionally substituted by one or more R₆;
R₆ is independently selected from the group consisting of hydroxy, C₁₋₁₀alkyl, C₁₋₁₀alkoxy, acyloxy, halo, nitro, amino, cyano, haloC₁₋₁₀alkyl, C₁₋₁₀alkylamino, diC₁₋₁₀alkylamino, acyl, and acyloxy;
R₇ is independently selected from the group consisting of C₁₋₁₀alkyl, C₂₋₁₀alkenyl, C₂₋₁₀alkynyl, C₁₋₁₀alkoxy, C₁₋₁₀alkoxycarbonylC₁₋₁₀alkyl, aryl, carboxyC₁₋₁₀alkyl, C₁₋₁₀alkylcarboxyC₁₋₁₀alkyl, C₁₋₁₀alkylcarboxyC₁₋₁₀aryl, heterocycle, hetercycleC₁₋₁₀alkyl, and heteroaryl, wherein any may be optionally substituted by one or more R₃; and
R₈ is independently selected from the group consisting of hydroxy, C₁₋₁₀alkyl, C₁₋₁₀alkoxy, acyloxy, halo, nitro, amino, cyano, and carboxy;
wherein two R₇ groups may come together to form a 4 to 7 membered ring.

In a narrower embodiment, the compound may be chosen from the formula Ib:

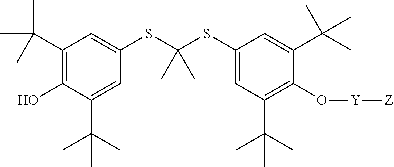

or a pharmaceutically acceptable salt wherein:
Y is a bond;
Z is selected from the group consisting of C₁₋₁₀alkyl, C₂₋₁₀alkenyl, C₂₋₁₀alkynyl, aryl, heteroaryl, C₁₋₁₀alkaryl, arylC₁₋₁₀alkyl, heteroarylC₁₋₁₀alkyl, C₁₋₁₀alkoxyC₁₋₁₀alkyl, C₁₋₁₀alkylaminoC₁₋₁₀alkyl, carboxyC₁₋₁₀alkyl, C₁₋₁₀dialkylaminoC₁₋₁₀alkyl, aminoC₁₋₁₀alkyl, heterocycle, R₇NH, carboxyC₁₋₁₀alkyl and R₇R₇N, wherein any may optionally be substituted by one or more R₅;
R₅ is independently selected from the group selected from hydroxy, C₁₋₁₀alkyl, C₁₋₁₀alkoxy, halo, nitro, amino, cyano, C₁₋₁₀alkylamino, diC₁₋₁₀alkylamino, acyl, acyloxy, COOH, COOR₇, OC(O)R₇, CH(OH)R₇, NHR₇, NR₇R₇, C(O)NH₂, C(O)NHR₇, CONR₇R₇, NHC(O)O—R₇, OSO₃H, SO₃H, SO₂NHR₇, SO₂NR₇R₇, P(O)(OH)OR₇, PO₂H₂ P(O)(OH)R₇, P(O)(OR₇)₂, P(O)R₇(OR₇), OPO₃H, PO₃H₂, hydroxymethyl, and cyclic phosphate, wherein when possible, all may be optionally substituted by one or more R₆;
R₆ is independently selected from the group consisting of hydroxy, C₁₋₁₀alkyl, C₁₋₁₀alkoxy, acyloxy, halo, nitro, amino, cyano, haloC₁₋₁₀alkyl, C₁₋₁₀alkylamino, diC₁₋₁₀alkylamino, acyl, and acyloxy;
R₇ is independently selected from the group consisting of C₁₋₁₀alkyl, C₂₋₁₀alkenyl, C₂₋₁₀alkynyl, C₁₋₁₀alkoxy, C₁₋₁₀alkoxycarbonylC₁₋₁₀alkyl, aryl, carboxyC₁₋₁₀alkyl, C₁₋₁₀alkylcarboxyC₁₋₁₀alkyl, C₁₋₁₀alkylcarboxyC₁₋₁₀aryl, heterocycle, hetercycleC₁₋₁₀alkyl, and heteroaryl, wherein any may be optionally substituted by one or more R₈; and
R₈ is independently selected from the group consisting of hydroxy, C₁₋₁₀alkyl, C₁₋₁₀alkoxy, acyloxy, halo, nitro, amino, cyano, and carboxy;
wherein two R₇ groups may come together to form a 4 to 7 membered ring.

In another embodiment of the above formula Ib:
Z is selected from the group consisting of C₁₋₆alkoxyC₁₋₆alkyl, and carboxyC₁₋₆alkyl, wherein any may optionally be substituted by one or more R₅;
R₅ is independently selected from the group selected from hydroxy, amino, halo, COOH, COOR₇, CH(OH)R₇, NHR₇, NR₇R₇, C(O)NH₂, C(O)NHR₇, CONR₇R₇, OSO₃H, SO₃H, SO₂NHR₇, SO₂NR₇R₇, P(O)(OH)OR₇, P(O)(OH)R₇, P(O)HR₇, P(OR₇)₂, P(O)R₇(OR₇), OPO₃H, PO₃H₂, and hydroxymethyl, wherein when possible, all may be optionally substituted by one or more R₆;
R₆ is independently selected from the group consisting of hydroxy, C₁₋₁₀alkyl, C₁₋₁₀alkoxy, acyloxy, halo, nitro, amino, cyano, haloC₁₋₁₀alkyl, C₁₋₁₀alkylamino, diC₁₋₁₀alkylamino, acyl, and acyloxy;
R₇ is independently selected from the group consisting of C₁₋₆alkyl, C₂₋₁₀alkenyl, C₂₋₆alkynyl, C₁₋₆alkoxy, C₁₋₆alkoxycarbonylC₁₋₆alkyl, carboxyC₁₋₆alkyl, and C₁₋₆alkylcarboxyC₁₋₆alkyl, wherein any may be optionally substituted by one or more R₈; and
R₈ is independently selected from the group consisting of hydroxy, C₁₋₆alkyl, C₁₋₆alkoxy, acyloxy, halo, amino, cyano, and carboxy.

In another embodiment of the above formula Ib:
Z is carboxyC₁₋₆alkyl, optionally substituted by one or more R₅;
R₅ is independently selected from the group consisting of halo, COOH, COOR₇, CONH₂, CONHR₇, CONR₇R₇, and amino;
R₇ is independently selected from the group consisting of C₁₋₆alkyl, carboxyC₁₋₆alkyl, C₁₋₆alkoxycarbonylC₁₋₆alkyl, and C₁₋₆alkylcarboxyC₁₋₆alkyl, wherein any may be optionally substituted by one or more R₈; and
R₈ is independently selected from the group consisting of hydroxy, halo, amino, and carboxy.

In another embodiment of the above formula Ib:
Z is carboxyC₁₋₆alkyl, optionally substituted by one or more R₅; and
R₅ is COOH.

Specific compounds of the above formula are

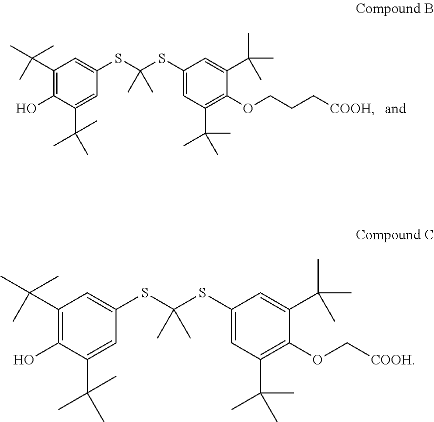

In yet another embodiment of the above formula Ib:
Z is selected from the group consisting of C₁₋₆alkyl, C₁₋₆alkoxyC₁₋₆alkyl, C₁-₆alkylaminoC₁-₆alkyl, C₁₋₆dialkylaminoC₁₋₆alkyl, and amino C₁₋₆alkyl, wherein any may optionally be substituted by one or more R₅;
R₅ is independently selected from the group selected from hydroxy, C₁₋₆alkyl, C₁₋₆alkoxy, acyloxy, halo, nitro, amino, cyano, C₁₋₆alkylamino, diC₁₋₆alkylamino, acyl, acyloxy, COOH, COOR₇, OC(O)R₇, CH(OH)R₇, NHR₇, NR₇R₇, C(O)NH₂, C(O)NHR₇, CONR₇R₇, NHC(O)O—R₇, OSO₃H, SO₃H, SO₂NHR₇, SO₂NR₇R₇, P(O)(OH)OR₇, P(O)HR₇, P(O)(OH)R₇, P(OR₇)₂, P(O)R₇(OR₇), OPO₃H, PO₃H₂, hydroxymethyl, and cyclic phosphate, wherein when possible, all may be optionally substituted by one or more R₆;
R₆ is independently selected from the group consisting of hydroxy, C₁₋₆alkyl, C₁₋₆alkoxy, acyloxy, halo, amino, cyano, haloC₁₋₆alkyl, C₁₋₆alkylamino, diC₁₋₆alkylamino, acyl, and acyloxy;
R₇ is independently selected from the group consisting of C₁₋₆alkyl, C₂₋₁₀alkenyl, C₂₋₁₀alkynyl, C₁₋₁₀alkoxy, C₁₋₁₀alkoxycarbonylC₁₋₁₀alkyl, carboxyC₁₋₆alkyl, C₁₋₆alkylcarboxyC₁₋₆alkyl, and heteroaryl, wherein any may be optionally substituted by one or more R₈; and
R₃ is independently selected from the group consisting of hydroxy, halo, amino, and carboxy.
   Specifically, the compound may be chosen from

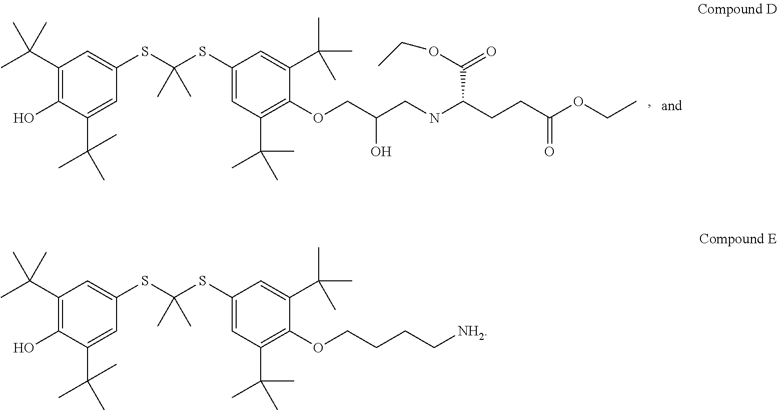

In another embodiment of the above formula Ib:
Z is selected from the group consisting of aryl, heteroaryl, C₁₋₁₀alkyl, C₁₋₆alkaryl, arylC₁₋₆alkyl, heteroarylC₁₋₆alkyl, and heterocycle, wherein any may optionally be substituted by one or more R₅;
R₅ is independently selected from the group selected from hydroxy, C₁₋₆alkyl, C₁₋₆alkoxy, acyloxy, halo, nitro, amino, cyano, C₁₋₆alkylamino, diC₁₋₆alkylamino, acyl, acyloxy, COOH, COOR₇, OC(O)R₇, CH(OH)R₇, NHR₇, NR₇R₇, C(O)NH₂, C(O)NHR₇, CONR₇R₇, NHC(O)O—R₇, OSO₃H, SO₃H, SO₂NHR₇, SO₂NR₇R₇, P(O)(OH)OR₇, P(O)HR₇, P(O)(OH)R₇, P(OR₇)₂, P(O)R₇(OR₇), OPO₃H, PO₃H₂, hydroxymethyl, and cyclic phosphate, wherein when possible, all may be optionally substituted by one or more R₆;
R₆ is independently selected from the group consisting of hydroxy, C₁₋₆alkyl, C₁₋₆alkoxy, acyloxy, halo, amino, cyano, haloC₁₋₆alkyl, C₁₋₆alkylamino, diC₁₋₆alkylamino, acyl, and acyloxy;
R₇ is independently selected from the group consisting of C₁₋₆alkyl, C₂₋₁₀alkenyl, C₂₋₁₀alkynyl, C₁₋₁₀alkoxycarbonylC₁₋₁₀alkyl, aryl, carboxyC₁₋₆alkyl, C₁₋₆alkylcarboxyC₁₋₆alkyl, C₁₋₆alkylcarboxyC₁₋₆aryl, heterocycle, hetercycleC₁₋₆alkyl, and heteroaryl, wherein any may be optionally substituted by one or more R₈; and
R₈ is independently selected from the group consisting of hydroxy, halo, amino, and carboxy;
wherein two R₇ groups may come together to form a 4 to 7 membered ring.

In another embodiment, the compound may be chosen from the following formula Ib:

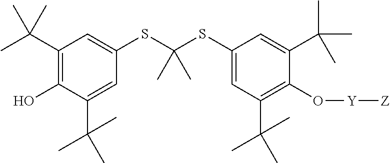

or a pharmaceutically acceptable salt wherein:
Y is

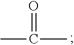

Z is selected from the group consisting of C₁₋₁₀alkyl, C₂₋₁₀alkenyl, C₂₋₁₀alkynyl, hydroxyC₁₋₁₀alkyl, aryl, heteroaryl, C₁₋₁₀alkaryl, arylC₁₋₁₀alkyl, heteroarylC₁₋₁₀alkyl, C₁₋₁₀alkoxyC₁₋₁₀alkyl, C₁₋₁₀alkylaminoC₁₋₁₀alkyl, carboxyC₁₋₁₀alkyl, C₁₋₁₀dialkylaminoC₁₋₁₀alkyl, aminoC₁₋₁₀alkyl, heterocycle, hetercycleC₁₋₁₀alkyl, R₇NH, R₇R₇N, carboxy, carbohydrate group, carbohydrate lactone group, and an alditol group wherein any may optionally be substituted by one or more R₅;
R₅ is independently selected from the group selected from hydroxy, C₁₋₁₀alkyl, C₁₋₁₀alkoxy, halo, nitro, amino, cyano, C₁₋₁₀alkylamino, diC₁₋₁₀alkylamino, acyl, acyloxy, COOH, COOR₇, OC(O)R₇, CH(OH)R₇, NHR₇, NR₇R₇, C(O)NH₂, C(O)NHR₇, CONR₇R₇, NHC(O)O—R₇, OSO₃H, SO₃H, SO₂NHR₇, SO₂NR₇R₇, P(O)(OH)OR₇, PO₂H₂ P(O)(OH)R₇, P(O)(OR₇)₂, P(O)R₇(OR₇), OPO₃H, PO₃H₂, hydroxymethyl, and cyclic phosphate, wherein when possible, all may be optionally substituted by one or more R₆;
R₆ is independently selected from the group consisting of hydroxy, C₁₋₁₀alkyl, C₁₋₁₀alkoxy, acyloxy, halo, nitro, amino, cyano, haloC₁₋₁₀alkyl, C₁₋₁₀alkylamino, diC₁₋₁₀alkylamino, acyl, and acyloxy;
R₇ is independently selected from the group consisting of C₁₋₁₀alkyl, C₂₋₁₀alkenyl, C₂₋₁₀alkynyl, C₁₋₁₀alkoxy, C₁₋₁₀alkoxycarbonylC₁₋₁₀alkyl, aryl, carboxyC₁₋₁₀alkyl, C₁₋₁₀alkylcarboxyC₁₋₁₀alkyl, C₁₋₁₀alkylcarboxyC₁₋₁₀aryl, heterocycle, hetercycleC₁₋₁₀alkyl, and heteroaryl, wherein any may be optionally substituted by one or more R₉; and
R₈ is independently selected from the group consisting of hydroxy, C₁₋₁₀alkyl, C₁₋₁₀alkoxy, acyloxy, halo, nitro, amino, cyano, and carboxy;
wherein two R₇ groups may come together to form a 4 to 7 membered ring.

In another embodiment of the above formula Ib:
Z is selected from the group consisting of C₁₋₆alkyl, hydroxyC₁₋₆alkyl, C₁₋₆alkoxyC₁₋₆alkyl, and carboxyC₁₋₆alkyl, wherein any may optionally be substituted by one or more R₅;
R₅ is independently selected from the group selected from hydroxy, amino, halo, COOH, COOR₇, CH(OH)R₇, NHR₇, NR₇R₇, C(O)NH₂, C(O)NHR₇, CONR₇R₇, OSO₃H, SO₃H, SO₂NHR₇, SO₂NR₇R₇, P(O)(OH)OR₇, P(O)(OH)R₇, P(O)HR₇, P(OR₇)₂, P(O)R₇(OR₇), OPO₃H, PO₃H₂, and hydroxymethyl, wherein when possible, all may be optionally substituted by one or more R₆;
R₇ is independently selected from the group consisting of C₁₋₆alkyl, C₂₋₁₀alkenyl, C₂₋₆alkynyl, C₁₋₆alkoxy, C₁₋₆alkoxycarbonylC₁₋₆alkyl, carboxyC₁₋₆alkyl, and C₁₋₆alkylcarboxyC₁₋₆alkyl, wherein any may be optionally substituted by one or more R₈; and
R₈ is independently selected from the group consisting of hydroxy, C₁₋₆alkyl, C₁₋₆alkoxy, acyloxy, halo, amino, cyano, and carboxy.

In another embodiment of the above formula Ib:
Z is C₁₋₆alkyl, optionally substituted by one or more R₅;
R₅ is independently selected from the group consisting of halo, COOH, COOR₇, CONH₂, CONHR₇, CONR₇R₇, and amino;
R₇ is independently selected from the group consisting of C₁₋₆alkyl, carboxyC₁₋₆alkyl, and C₁₋₆alkylcarboxyC₁₋₆alkyl, wherein any may be optionally substituted by one or more R₈; and
R₈ is independently selected from the group consisting of hydroxy, halo, amino, and carboxy.

In another embodiment of the above formula Ib:
Z is C₁₋₆alkyl, optionally substituted by one or more R₅; and
R₅ is COOH.

Specifically, the compound may be chosen from

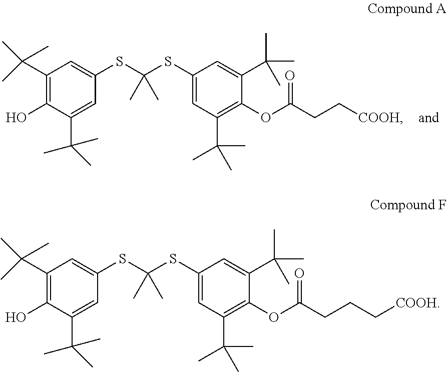

In another embodiment of the above formula Ib:
Z is selected from the group consisting of C₁₋₆alkyl, C₁₋₆alkoxyC₁₋₆alkyl, C₁-₆alkylaminoC₁-₆alkyl, and aminoC₁₋₆alkyl, wherein any may optionally be substituted by one or more R₅;
R₅ is independently selected from the group selected from hydroxy, C₁₋₆alkyl, C₁₋₆alkoxy; acyloxy, halo, nitro, amino, cyano, C₁₋₆alkylamino, diC₁₋₆alkylamino, acyl, acyloxy, COOH, COOR₇, OC(O)R₇, CH(OH)R₇, NHR₇, NR₇R₇, C(O)NH₂, C(O)NHR₇, CONR₇R₇, NHC(O)O—R₇, OSO₃H, SO₃H, SO₂NHR₇, SO₂NR₇R₇, P(O)(OH)OR₇, P(O)HR₇, P(O)(OH)R₇, P(OR₇)₂, P(O)R₇(OR₇), OPO₃H, PO₃H₂, hydroxymethyl, and cyclic phosphate, wherein when possible, all may be optionally substituted by one or more R₆;
R₆ is independently selected from the group consisting of hydroxy, C₁₋₆alkyl, C₁₋₆alkoxy, acyloxy, halo, amino, cyano, C₁₋₆alkylamino, diC₁₋₆alkylamino, acyl, and acyloxy;
R₇ is independently selected from the group consisting of C₁₋₆alkyl, C₂₋₁₀alkenyl, C₂₋₁₀alkynyl, C₁₋₁₀alkoxy, C₁₋₁₀alkoxycarbonylC₁₋₁₀alkyl, carboxyC₁₋₆alkyl, C₁₋₆alkylcarboxyC₁₋₆alkyl, and heteroaryl, wherein any may be optionally substituted by one or more R₈; and
R₈ is independently selected from the group consisting of hydroxy, halo, amino, and carboxy

In another embodiment of the above formula Ib, the compound may be

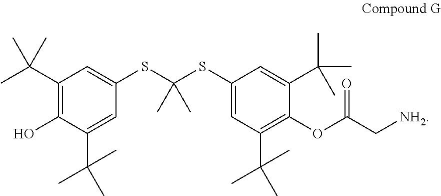

In another embodiment of the above formula:
Z is selected from the group consisting of C₁₋₆alkyl, aryl, heteroaryl, C₁₋₆alkaryl, arylC₁₋₆alkyl, heteroarylC₁₋₆alkyl, heterocycle, and hetercycleC₁₋₆alkyl, wherein any may optionally be substituted by one or more R₅;
R₅ is independently selected from the group selected from hydroxy, C₁₋₆alkyl, C₁₋₆alkoxy, acyloxy, halo, nitro, amino, cyano, C₁₋₆alkylamino, diC₁₋₆alkylamino, acyl, acyloxy, COOH, COOR₇, OC(O)R₇, CH(OH)R₇, NHR₇, NR₇R₇, C(O)NH₂, C(O)NHR₇, CONR₇R₇, NHC(O)O—R₇, OSO₃H, SO₃H, SO₂NHR₇, SO₂NR₇R₇, P(O)(OH)OR₇, P(O)HR₇, P(O)(OH)R₇, P(OR₇)₂, P(O)R₇(OR₇), OPO₃H, PO₃H₂, hydroxymethyl, and cyclic phosphate, wherein when possible, all may be optionally substituted by one or more R₆;
R₆ is independently selected from the group consisting of hydroxy, C₁₋₆alkyl, C₁₋₆alkoxy, acyloxy, halo, amino, cyano, C₁₋₆alkylamino, diC₁₋₆alkylamino, acyl, and acyloxy;
R₇ is independently selected from the group consisting of C₁₋₆alkyl, C₂₋₁₀alkenyl, C₂₋₁₀alkynyl, C₁₋₁₀alkoxy, C₁₋₁₀alkoxycarbonylC₁₋₁₀alkyl, aryl, carboxyC₁₋₆alkyl, C₁₋₆alkylcarboxyC₁₋₆alkyl, C₁₋₆alkylcarboxyC₁₋₆aryl, heterocycle, hetercycleC₁₋₆alkyl, and heteroaryl, wherein any may be optionally substituted by one or more R₈; and
R₈ is independently selected from the group consisting of hydroxy, halo, amino, and carboxy;
wherein two R₇ groups may come together to form a 4 to 7 membered ring.

Other compounds contemplated for use in the methods and compositions disclosed herein are:

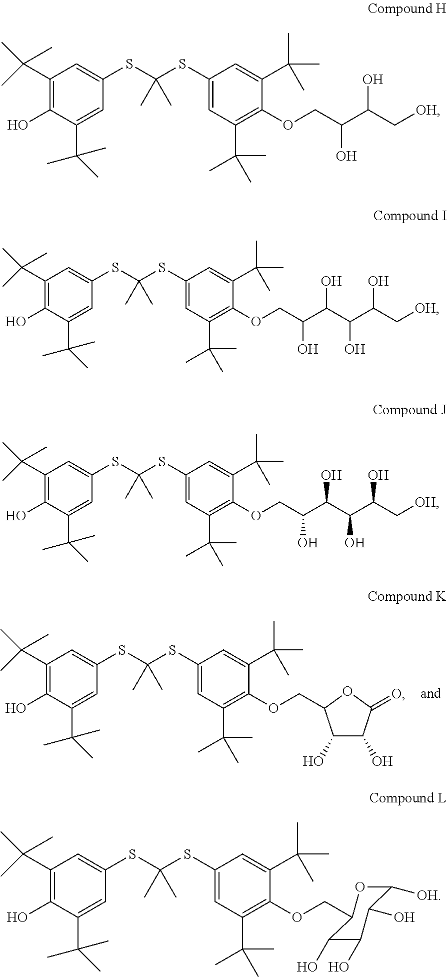

In another embodiment, the compound is a compound of Formula I:

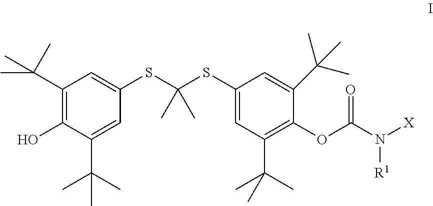

wherein
R¹ is independently hydrogen or C₁-C₄ alkyl ; and
X is independently C₁-C₄ alkyl, optionally substituted by one or more hydroxyl or C(O)OH, which are optionally protected.

In one embodiment, the one or more hydroxyl or C(O)OH groups are protected.

Optionally, X is C₁-C₄ alkyl substituted by two or more hydroxyl groups.

Optionally, X is C₁-C₄ alkyl substituted by three or more hydroxyl groups.

Optionally, R¹ is substituted C₁-C₄ alkyl, e.g., substituted with a halogen, such as fluoro.

X is, e.g., a C₁, C₂, C₃ or C₄ alkyl.

Specific embodiments include:

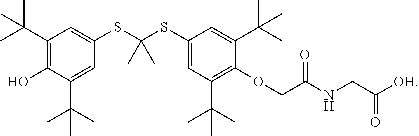

(2-{2,6-Di-tert-butyl-4-[1-(3,5-di-tert-butyl-4-hydroxyphenylsulfanyl)-1-methylethylsulfanyl]phenoxy}acetylamino)acetic acid;

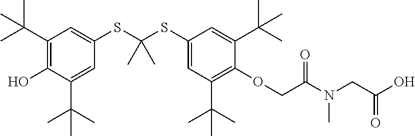

[(2-{2,6-Di-tert-butyl-4-[1-(3,5-di-tert-butyl-4-hydroxyphenylsulfanyl)-1-methylethylsulfanyl]phenoxy}acetyl)methylamino]acetic acid;

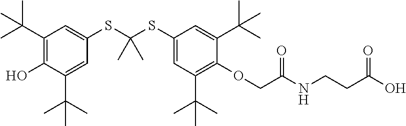

3-(2-{2,6-Di-tert-butyl-4-[1-(3,5-di-tert-butyl-4-hydroxyphenylsulfanyl)-1-methylethylsulfanyl]phenoxy}acetylamino)propionic acid; and

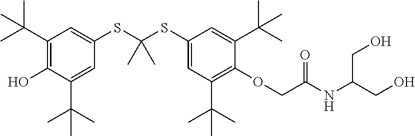

2-{2,6-Di-tert-butyl-4-[1-(3,5-di-tert-butyl-4-hydroxyphenylsulfanyl)-1-methyl-ethylsulfanyl]phenoxy}-N-(2-hydroxy-1-hydroxymethyl-ethyl)acetamide.

In one embodiment, the compound is a compound of Formula II:

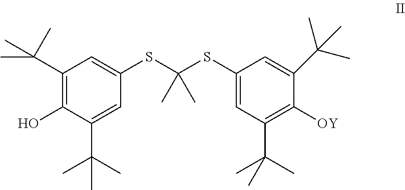

wherein Y is selected from the group consisting of:

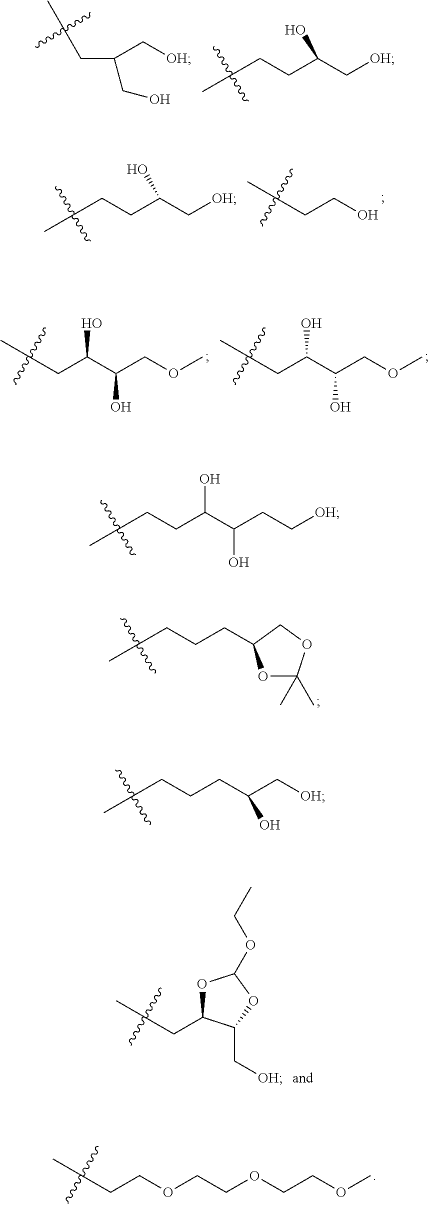

The compounds specifically include the following:

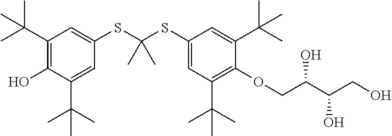

4-{2,6-Di-tert-butyl-4-[1-(3,5-di-tert-butyl-4-hydroxyphenylsulfanyl)-1-methylethylsulfanyl]phenoxy}butane-1,2(S),3(S)-triol;

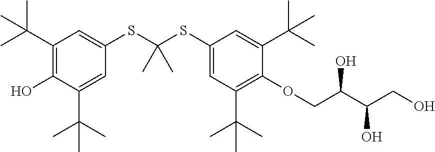

4-{2,6-Di-tert-butyl-4-[1-(3,5-di-tert-butyl-4-hydroxyphenylsulfanyl)-1-methylethylsulfanyl]-phenoxy}butane-1,2(R),3(R)-triol;

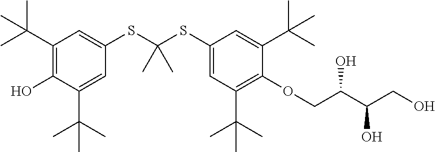

4-{2,6-Di-tert-butyl-4-[1-(3,5-di-tert-butyl-4-hydroxyphenylsulfanyl)-1-methylethylsulfanyl]-phenoxy}butane-1,2(S),3(R)-triol; and

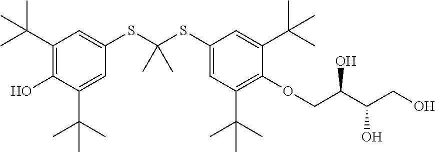

4-{2,6-Di-tert-butyl-4-[1-(3,5-di-tert-butyl-4-hydroxyphenylsulfanyl)-1-methylethylsulfanyl]-phenoxy}butane-1,2(R),3(S)-triol.

In yet another embodiment, the compound is a compound of Formula II above wherein Y is selected from the group consisting of:

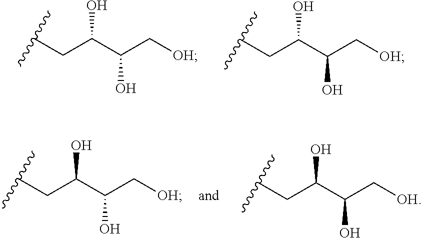

### Stereochemistry

It is appreciated that compounds having a chiral center may exist in and be isolated in optically active and racemic forms. Some compounds may exhibit polymorphism. It is to be understood that the present invention encompasses any racemic, optically-active, diastereomeric, polymorphic, or stereoisomeric form, or mixtures thereof, of a compound, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase).

Examples of methods to obtain optically active materials are known in the art, and include at least the following.
i) physical separation of crystals—a technique whereby macroscopic crystals of the individual enantiomers are manually separated. This technique can be used if crystals of the separate enantiomers exist, i.e., the material is a conglomerate, and the crystals are visually distinct;
ii) simultaneous crystallization—a technique whereby the individual enantiomers are separately crystallized from a solution of the racemate, possible only if the latter is a conglomerate in the solid state;
iii) enzymatic resolutions—a technique whereby partial or complete separation of a racemate by virtue of differing rates of reaction for the enantiomers with an enzyme;
iv) enzymatic asymmetric synthesis—a synthetic technique whereby at least one step of the synthesis uses an enzymatic reaction to obtain an enantiomerically pure or enriched synthetic precursor of the desired enantiomer;
v) chemical asymmetric synthesis—a synthetic technique whereby the desired enantiomer is synthesized from an achiral precursor under conditions that produce asymmetry (i.e., chirality) in the product, which may be achieved using chiral catalysts or chiral auxiliaries;
vi) diastereomer separations—a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;
vii) first- and second-order asymmetric transformations—a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;
viii) kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;
ix) enantiospecific synthesis from non-racemic precursors—a synthetic technique whereby the desired enantiomer is obtained from non-chiral starting materials and where the stereochemical integrity is not or is only minimally compromised over the course of the synthesis;
x) chiral liquid chromatography—a technique whereby the enantiomers of a racemate are separated in a liquid mobile phase by virtue of their differing interactions with a stationary phase. The stationary phase can be made of chiral material or the mobile phase can contain an additional chiral material to provoke the differing interactions;
xi) chiral gas chromatography—a technique whereby the racemate is volatilized and enantiomers are separated by virtue of their differing interactions in the gaseous mobile phase with a column containing a fixed non-racemic chiral adsorbent phase;
xii) extraction with chiral solvents—a technique whereby the enantiomers are separated by virtue of preferential dissolution of one enantiomer into a particular chiral solvent;
xiii) transport across chiral membranes—a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane which allows only one enantiomer of the racemate to pass through.

Some of the compounds may exist in tautomeric, geometric or stereoisomeric forms. The present invention contemplates all such compounds, including cis- and trans-geometric isomers, E- and Z-geometric isomers, R- and S-enantiomers, diastereomers, d-isomers, l-isomers, the racemic mixtures thereof and other mixtures thereof, as falling within the scope of the invention. Pharmaceutically acceptable sales of such tautomeric, geometric or stereoisomeric are also included within the invention. The terms “cis” and “trans” denote a form of geometric isomerism in which two carbon atoms connected by a double bond will each have two high ranking groups on the same side of the double bond (“cis”) or on opposite sides of the double bond (“trans”). Some of the compounds described contain alkenyl groups, and are meant to include both cis and trans or “E” and “Z” geometric forms. Some of the compounds described contain one or more stereocenters and are meant to include R, S, and mixtures of R and S forms for each stereocenter present.

Some of the compounds described herein may contain one or more ketonic or aldehydic carbonyl groups or combinations thereof alone or as part of a heterocyclic ring system. Such carbonyl groups may exist in part or principally in the “keto” form and in part or principally as one or more “enol” forms of each aldehyde and ketone group present. Compounds having aldehydic or ketonic carbonyl groups are meant to include both “keto” and “enol” tautomeric forms.

Some of the compounds described herein may contain one or more imine or enamine groups or combinations thereof. Such groups may exist in part or principally in the “imine” form and in part or principally as one or more “enamine” forms of each group present. Compounds having said imine or enamine groups are meant to include both “imine” and “enamine” tautomeric forms.

### Therapeutic Methods

The phenolic antioxidant compounds described herein, or their pharmaceutically acceptable salts, esters or prodrugs, are useful for reducing or inhibiting platelet activation or aggregation, or reducing thrombin levels, or reducing the activity or expression of thrombin receptors. The invention therefore provides a new approach for the treatment and/or prevention of thrombotic or thromboembolic disorders accompanied or characterized by platelet activation, elevated thrombin levels or increased expression of thrombin receptors.

In one embodiment, compounds or compositions comprising the compounds are administered in an effective amount to reduce platelet activation in an individual, or to inhibit thrombin formation in an individual.

In one embodiment, a method of reducing a platelet activation state of an individual in need thereof is provided, the method comprising administering an effective amount of a compound as disclosed herein.

In another embodiment, a method of treating an individual in need thereof is provided, the method comprising administering an effective amount of a compound disclosed herein to inhibit platelet aggregation, for example by at least 10%.

In another embodiment, a method of treating an individual in need thereof is provided, the method comprising administering an effective amount of a compound disclosed herein to inhibit platelet aggregation as measured by the level of platelet PAR-1/PAR-4 thrombin receptor expression.

The compound in one embodiment reduces the activity of protease activating receptors 1 or 4 (PAR-1 or PAR-4) in platelets of the individual. Optionally, the method includes comparing the level of platelet PAR-1/PAR-4 thrombin receptor expression from a sample taken from the individual after administration of the compound to a control, and detecting a decrease in the expression.

In another embodiment, the method includes administering an effective amount of the phenolic antioxidant compound to reduce the levels of at least one platelet activation marker in an individual. The method may include comparing the level of at least one platelet activation marker from a sample taken from the individual, to a control, wherein the level is decreased after administration of the compound, for example by at least about 10%, 15%, 17%, or 20% or more. The level of two, three, four, five, six or more platelet activation markers may be reduced.

The compound is, for example, administered, optionally in a pharmaceutically acceptable carrier, orally, intravenously, intramuscularly, intraperitoneally, topically, sublingually, subcutaneously, parenterally, transdermally, intradermally, intraocularly, intranasally, by inhalation, by implant, or by suppository. In one embodiment, the compound is administered orally in an amount between about 0.5 mg-2500 mg/daily. In another embodiment, the compound is administered orally in an amount between about 1 mg-2500 mg/daily, 5 mg-2500 mg/daily, 5 mg-1000 mg/daily, 5 mg-500 mg/daily, 10 mg-250 mg/daily, or 10 mg-200 mg/daily.

In particular embodiments, the compounds may be used for the treatment er prevention of angina, myocardial infarction, stroke, pulmonary embolism, transient ischemic attack, coronary ischemic syndrome, Syndrome X, heart failure, diabetes, and disorders in which a narrowing of at least one coronary artery occurs. The compounds also may be used to treat or prevent thrombosis including catheter thrombosis, deep vein thrombosis, arterial vessel thrombosis, and peripheral vascular thrombosis. The compounds further may be used for the treatment of thrombotic occlusion and re-occlusion, including re-occlusion subsequent to a coronary intervention procedure, or in connection with heart surgery or vascular surgery.

In another embodiment, the compounds can be used for the treatment of thromboembolisms, including venous thromboembolism in elective orthopedic replacement surgery, such as knee or hip replacement surgery, or other surgeries. The compounds can be used for the reduction or prevention of stroke in patients with atrial fibrillation, and for the prevention of secondary vascular events after myocardial infarction.

Optionally, the compound may be administered after thrombolysis has occurred. The method may include treating humans or animals.

The term, “thrombotic or thromboembolic event,” includes any disorder that involves a blockage or partial blockage of an artery or vein with a thrombosis or thromboembolism, all of which can be treated by the compounds disclosed herein. A “thrombosis” is the formation of a clot (or thrombus) inside a blood vessel, that can obstruct the flow of blood through the circulatory system. A “thromboembolism” involves formation in a blood vessel of a clot (thrombus) that breaks loose and is carried by the blood stream to lodge in another vessel area. The clot may lodge in a vessel in the lungs (pulmonary embolism), brain (stroke), gastrointestinal tract, kidneys, or leg. Thromboembolism is an important cause of morbidity (disease) and mortality (death), especially in adults.

A thrombotic or thromboembolic event occurs when a clot forms and lodges within a blood vessel. The clot may fully or partially block the blood vessel causing a thrombotic disorder such as a heart attack or stroke. Examples of thrombotic or thromboembolic events include thrombotic disorders such as acute myocardial infarction, unstable angina, ischemic stroke, acute coronary syndrome, pulmonary embolism, transient ischemic attack, thrombosis (e.g. deep vein thrombosis, thrombotic occlusion and re-occlusion and peripheral vascular thrombosis) and thromboembolism. A thrombotic or thromboembolic event also includes first or subsequent thrombotic stroke, acute myocardial infarction, which occurs subsequent to a coronary intervention procedure, or thrombolytic therapy.

The compound can be administered e.g., intravenously, parenterally, orally, subcutaneously, intramuscularly, transdermally (for example using an iontophoretic patch), intraocularly, intranasally, by inhalation, by implant, by suppository, or by other routes known to those skilled in the medical arts, taking into account the particular properties of the compound being administered and the particular therapy.

In one embodiment, the compound is administered about 6 hours to 24 hours after thrombolysis has occurred, about 12 hours to 24 hours after thrombolysis has occurred, or about 20 hours to 24 hours after thrombolysis has occurred. In another aspect, the compound is administered multiple times. The number of doses administered will depend on the type and severity of the thrombotic or thromboembolic condition to be treated. This determination can be made by one skilled in the art and is within the scope of the invention.

In one embodiment, the compound may be administered on an ongoing basis to treat or prevent angina, myocardial infarction, stroke, pulmonary embolism, transient ischemic attack, coronary ischemic syndrome, Syndrome X, heart failure, diabetes, disorders in which a narrowing of at least one coronary artery occurs, thrombosis including catheter thrombosis, deep vein thrombosis, arterial vessel thrombosis, and peripheral vascular thrombosis, or thrombotic occlusion and re-occlusion, including re-occlusion subsequent to a coronary intervention procedure, or in connection with heart surgery or vascular surgery.

Therapeutically effective amounts of the compounds are suitable for use in the compositions and methods of the present invention. The dosage regimen is selected in accordance with a variety of factors including type, species, age, weight, sex and medical condition of the patient; the severity of the condition to be treated; the route of administration; the renal and hepatic function of the patient; and the particular compound or salt or ester thereof employed. In one embodiment, the compound is administered orally in an amount of about 0.5-2500 mg/day. In subembodiment, the compound is administered orally in an amount between about 1 mg-2500 mg/daily, 5 mg-2500 mg/daily, 5 mg-1000 mg/daily, 5 mg-500 mg/daily, 10 mg-250 mg/daily, or 10 mg-200 mg/daily.

### Assays for Compound Activity

A number of assays are known in the art for measuring thrombolysis and components of the thrombolytic system. For example, U.S. Pat. No. 5,612,187 provides a clot time determining device and method for determining the time necessary for a test fluid to lyse a clot.

Coagulation assay procedures are described in, for example, Smith, et al., (Smith et al. (1988) Thrombosis Research, 50:163-174). U.S. Pat. Nos. 5,688,813 and 5,668,289 teach assaying coagulation time and related determinations in murine and canine models of arterial injury and of coronary artery thrombosis. U.S. Pat. Nos. 4,861,712; 4,910,510; 5,059,525; and 5,580,744 describe test articles suitable for monitoring blood coagulation. U.S. Pat. No. 4,756,884 describes a capillary flow device for measuring blood characteristics, including prothrombin time. A platelet aggregation assay, a platelet-fibrinogen binding assay, and a thrombolytic assay, are all taught in U.S. Pat. No. 5,661,159. Simple tests, such as rocking a blood sample in a test tube and timing the period until the blood clots, in the presence or absence of known or potential anti-coagulants, as well as whole blood aggregation techniques and flow cytometric analysis of appropriate adhesion surface markers are also known. Whichever assay is employed, the assays can be performed serially and as often as possible in order to assure accurate measurements.

Success of thrombolysis can be determined by using a number of techniques that are well known in the art. For example, thrombolysis can be evaluated by angiography, scintigraphy, electrocardiogram (ECG), patient condition (i.e. assessment of symptom relief) and, indirectly, by measuring the plasma levels of myocardial necrosis biomarkers.

Methods for Measuring Platelet Aggregation and Platelet Activation Markers

The following studies can be performed in human subjects or laboratory animal models, such as mice. Prior to the initiation of a clinical study involving human subjects, the study should be approved by the appropriate Human Subjects Committee and subjects should be informed about the study and give written consent prior to participation.

The compound therapy using a selected compound disclosed herein can be evaluated in comparison to a control treatment such as a placebo treatment. Th5 dosages may be readily determined by a skilled artisan conducting the study. The length of the study treatment will vary on a particular study and can also be determined by one of ordinary skill in the art. By way of example, the therapy may be administered for 4 weeks. The compound can be administered by any route as described herein, e.g. administration orally for human subjects.

Platelet activation can be determined by a number of tests available in the art. Several such tests are described below. In order to determine the effectiveness of the treatment, the state of platelet activation is evaluated at several time points during the study, such as before administering the combination treatment and once a week during treatment. The exemplary procedures for blood sampling and the analyses that can be used to monitor platelet aggregation are listed below.

A. Platelet Aggregation Study

Blood samples are collected from an antecubital vein via a 19-gauge needle into two plastic tubes. Each sample of free flowing blood is collected through a fresh venipuncture site distal to any intravenous catheters using a needle and Vacutainer hood into 7 cc Vacutainer tubes (one with CTAD (dipyridamole), and the other with 3.8% trisodium citrate). If blood is collected simultaneously for any other studies, it is preferable that the platelet sample be obtained second or third, but not first. If only the platelet sample is collected, the initial 2-3 cc of blood is discharged and then the vacutainer tube is filled. The venipuncture is adequate if the tube fills within 15 seconds. All collections are performed by trained personnel.

After the blood samples for each subject have been collected into two Vacutainer tubes, they are immediately, but gently, inverted 3 to 5 times to ensure complete mixing of the anticoagulant. Tubes are not shaken. The Vacutainer tubes are filled to capacity, since excess anticoagulant can alter platelet function. Attention is paid to minimizing turbulence whenever possible. Small steps, such as slanting the needle in the Vacutainer to have the blood run down the side of tube instead of shooting all the way to the bottom, can result in significant improvement. These tubes are kept at room temperature and transferred directly to the laboratory personnel responsible for preparing the samples. The Vacutainer tubes are not chilled at any time.

Trisodium citrate (3.8%) and whole blood is immediately mixed in a 1:9 ratio, and then centrifuged at 1200 g for 2.5 minutes, to obtain platelet-rich plasma (PRP), which is kept at room temperature for use within 1 hour for platelet aggregation studies. Platelet count is determined in each PRP sample with a Coulter Counter ZM (Coulter Co., Hialeah, Fla.). Platelet numbers are adjusted to 3.50×10⁸/ml for aggregation with homologous platelet-poor plasma. PRP and whole blood aggregation tests are performed simultaneously. Whole blood is diluted 1:1 with the 0.5 ml PBS, and then swirled gently to mix. The cuvette with the stirring bar is placed in the incubation well and allowed to warm to 37° C. for 5 minutes. Then the samples are transferred to the assay well. An electrode is placed in the sample cuvette. Platelet aggregation is stimulated with 5 μM ADP, 1 μg/ml collagen, and 0.75 mM arachidonic acid. All agonists are obtained, e.g., from Chronolog Corporation (Hawertown, Pa.). Platelet aggregation studies are performed using a Chrono-Log Whole Blood Lumi-Aggregometer (model 560-Ca). Platelet aggregability is expressed as the percentage of light transmittance change from baseline using platelet-poor plasma as a reference at the end of recording time for plasma samples, or as a change in electrical impedance for whole blood samples. Aggregation curves are recorded for 4 minutes and analyzed according to internationally established standards using Aggrolink® software.

Aggregation curves of subjects receiving compound therapy can then be compared to the aggregation curves of subjects receiving a control treatment in order to determine the efficacy of the therapy.

B. Washed Platelets Flow Cytometry

Venous blood (8 ml) is collected in a plastic tube containing 2 ml of acid-citrate-dextrose (ACD) (7.3 g citric acid, 22.0 g sodium citrate.2H₂O and 24.5 glucose in 1000 ml distilled water) and mixed well. The blood-ACD mixture is centrifuged at 1000 r.p.m. for 10 minutes at room temperature. The upper ⅔ of the platelet-rich plasma (PRP) is then collected and adjusted to pH=6.5 by adding ACD. The PRP is then centrifuged at 3000 r.p.m. for 10 minutes. The supernatant is removed and the platelet pellet is gently resuspended in 4 cc of the washing buffer (10 mM Tris/HCl, 0.15 M NaCl, 20 mM EDTA, pH=7.4). Platelets are washed in the washing buffer, and in TBS (10 mM Tris, 0.15 M NaCl, pH=7.4). All cells are then divided into the appropriate number of tubes. By way of example, if 9 different surface markers are evaluated, as described herein, then the cells should be divided into ten tubes, such that nine tubes containing washed platelets are incubated with 5 μl fluorescein isothiocyanate (FITC)-conjugated antibodies in the dark at +4° C. for 30 minutes, and one tube remains unstained and serves as a negative control. Surface antigen expression is measured with monoclonal murine anti-human antibodies, such as CD9 (p24); CD41a (IIb/IIIa, aIIbb3); CD42b (Ib); CD61(IIIa) (DAKO Corporation, Carpinteria, Calif.); CD49b (VLA-2, or a2b1); CD62p (P-selectin); CD31 (PECAM-1); CD 41b (IIb); and CD51/CD61 (vitronectin receptor, avb3) (PharMingen, San Diego Calif.), as the expression of these antigens on the cells is associated with platelet activation. After incubation, the cells are washed with TBS and resuspended in 0.25 ml of 1% paraformaldehyde. Samples are stored in the refrigerator at +4° C., and analyzed on a Becton Dickinson FACScan flow cytometer with laser output of 15 mw, excitation at 488 nm, and emission detection at 530±30 nm. The data can be collected and stored in list mode, and then analyzed using CELLQuest® software. FACS procedures are described in detail in, e.g., Gurbel, P. A. et al., J Amer Coll Cardiol 31: 1466-1473 (1998); Serebruany, V. L. et al., Am Heart J 136: 398-405 (1998); Gurbel, P. A. et al., Coron Artery Dis 9: 451-456 (1998) and Serebruany, V. L. et al., Arterioscl Thromb Vasc Biol 19: 153-158 (1999).

The antibody staining of platelets isolated from subjects receiving a combination therapy can then be compared to the staining of platelets isolated from subjects receiving a control treatment in order to determine the effect of the combination therapy on platelets. C. Whole Blood Flow Cytometry

Four cc of blood is collected in a tube, containing 2 cc of acid-citrate-dextrose (ACD; 7.3 g citric acid, 22.0 g sodium citrate.2H₂O and 24.5 glucose in 1000 ml distilled water) and mixed well. The buffer, TBS (10 mM Tris, 0.15 M NaCl, pH 7.4) and the following fluorescein isothiocyanate (FITC) conjugated monoclonal antibodies (PharMingen, San Diego, Calif., USA, and DAKO, Calif., USA) are removed from a refrigerator and allowed to warm at room temperature (RT) prior to their use. The non-limiting examples of antibodies that can be used include CD41 (IIb/IIIa), CD31 (PECAM-1), CD62p (P-selectin), and CD51/61 (Vitronectin receptor). For each subject, six amber tubes (1.25 ml) are one Eppendorf tube (1.5 ml) are obtained and marked appropriately 450 ml of TBS buffer is pipetted to the labeled Eppendorf tube. A patient's whole blood tube is inverted gently twice to mix, and 50 ml of whole blood is pipetted to the appropriately labeled Eppendorf tube. The Eppendorf tube is capped and the diluted whole blood is mixed by inverting the Eppendorf tube gently two times, followed by pipetting 50 μl of diluted whole blood to each amber tube. 5 pl of appropriate antibody is pipetted to the bottom of the corresponding amber tube. The tubes are covered with aluminum foil and incubated at 4° C. for 30 minutes. After incubation, 400 μl of 2% buffered paraformaldehyde is added. The amber tubes are closed with a lid tightly and stored in a refrigerator at 4° C. until the flow cytometric analysis. The samples are analyzed on a Becton Dickinson FACScan flow cytometer. These data are collected in list mode files and then analyzed. The antibody staining of platelets isolated from subjects receiving compound therapy can then be compared to the staining of platelets isolated from subjects receiving a control treatment.

D. ELISA

Enzyme-linked immunosorbent assays (ELISA) are used according to standard techniques and as described herein. Eicosanoid metabolites may be used to determine platelet aggregation. The metabolites are analyzed due to the fact that eicosanoids have a short half-life under physiological conditions. Thromboxane B2 (TXB₂), the stable breakdown product of thromboxane A₂ and 6keto-PGF₁ alpha, the stable degradation product of prostacyclin may be tested. Thromboxane B2 is a stable hydrolysis product of TXA₂ and is produced following platelet aggregation induced by a variety of agents, such as thrombin and collagen. 6keto-prostaglandin F₁ alpha is a stable hydrolyzed product of unstable PGI₂ (prostacyclin). Prostacyclin inhibits platelet aggregation and induces vasodilation. Thus, quantitation of prostacyclin production can be made by determining the level of 6keto-PGF₁. The metabolites may be measured in the platelet poor plasma (PPP), which is kept at −4° C. Also, plasma samples may also be extracted with ethanol and then stored at −80° C. before final prostaglandin determination, using, e.g., TiterZymes® enzyme immunoassays according to standard techniques (PerSeptive Diagnostics, Inc., Cambridge, Mass., USA). ELISA kits for measuring TXB₂ and 6keto-PGF₁ are also commercially available.

The amounts of TXB₂ and 6keto-PGF₁ in plasma of subjects receiving compound therapy and subjects receiving a control can be compared to determine the efficacy of the treatment.

E. Closure Time Measured with the Dade Behring Platelet Function Analyzer, PFA-100®

PFA-100® can be used as an in vitro system for the detection of platelet dysfunction. It provides a quantitative measure of platelet function in anticoagulate whole blood. The system comprises a microprocessor-controlled instrument and a disposable test cartridge containing a biologically active membrane. The instrument aspirates a blood sample under constant vacuum from the sample reservoir through a capillary and a microscopic aperture cut into the membrane. The membrane is coated with collagen and epinephrine or adenosine 5′-diphosphate. The presence of these biochemical stimuli, and the high shear rates generated under the standardized flow conditions, result in platelet attachment, activation, and aggregation, slowly building a stable platelet plug at the aperture. The time required to obtain full occlusion of the aperture is reported as the “closure time,” which normally ranges from one to three minutes.

The membrane in the PFA-100® test cartridge serves as a support matrix for the biological components and allows placement of the aperture. The membrane is a standard nitrocellulose filtration membrane with an average pore size of 0.45 μm. The blood entry side of the membrane was coated with 2 μg of fibrillar Type I equine tendon collagen and 10 .mu.g of epinephrine bitartrate or 50 μg of adenosine 5′-diphosphate (ADP). These agents provide controlled stimulation to the platelets as the blood sample passes through the aperture. The collagen surface also served as a well-defined matrix for platelet deposition and attachment.

The principle of the PFA-100® test is very similar to that described by Kratzer and Born (Kratzer, et al., Haemostasis 15: 357-362 (1985)). The test utilizes whole blood samples collected in 3.8% of 3.2% sodium citrate anticoagulant. The blood sample is aspirated through the capillary into the cup where it comes in contact with the coated membrane, and then passes through the aperture. In response to the stimulation by collagen and epinephrine or ADP present in the coating, and the shear stresses at the aperture, platelets adhere and aggregate on the collagen surface starting at the area surrounding the aperture. During the course of the measurement, a stable platelet plug forms that ultimately occludes the aperture. The time required to obtain full occlusion of the aperture is defined as the “closure time” and is indicative of the platelet function in the sample. Accordingly, “closure times” can be compared between subjects receiving a compound therapy and the ones receiving a control therapy in order to evaluate the efficacy of the treatment.

It should be noted that all of the above-mentioned procedures can be modified for a particular study, depending on factors such as a drug used, length of the study, subjects that are selected, etc. Such modifications can be designed by a skilled artisan without undue experimentation.

### Combination or Alternation Therapy

The above compounds may be administered alone or in combination or alternation with one or more therapeutic drugs, including any drugs having anticoagulant, anti-thrombin, thrombolytic, fibrinolytic or anti-platelet activity.

Anti-platelet compounds that can be used include:
platelet adhesion inhibitors, such as aspirin, dipyridamole (Persantine®, Boehringer Ingelheim), or Aggrenox® (aspirin-diypridamole);
a glycoprotein IIb/IIIa inhibitor, such as abciximab (ReoPro®, Eli Lilly & Co.), eptifibatide (Integrilin®, Cor Therapeutics), tirofoban (Aggrastat®, Merck & Co., Inc.), roxifiban, and lamifiban; and
inhibitors of ADP-induced platelet activation, including thienopyridines, such as clopidogrel (Plavix®, Sanofi-Bristol Myers Squibb), ticlopidine (Ticlid®, Roche Laboratories) and prasugrel (Eli Lilly).

The second therapeutic agent also can be an anticoagulant compound. Non-limiting examples of anti-coagulant compounds include heparin, such as unfractionated heparin, or low molecular weight heparins, such as enoxaparin (Lovenox®; Sanofi-Aventis), dalteparin (Fragmin®; Pharmacia & Upjohn), tinzaparin (Innohep®; Dupont), and ardeparin (Normiflo®; Wyeth-Ayerst Laboratories). Other options include warfarin (Coumadin) and hirudin.

Thrombin inhibitors that can be used include danaproid (Orgaran), lepirudin (Refludan®; Berlex Laboratories), bivalirudin (Angiomax®; The Medicines Company) argatroban (Glaxo Smith Kline).

Thrombolytic agents can be used including recombinant tissue plasminogen activator, such as alteplase (also known as t-PA or Activase®, Genentech, Inc.), other forms of tissue plasminogen activator, such as reteplase (also known as r-PA or retavase®, Centocor, Inc.) and tenecteplase (also known as TNK®, Genentech, Inc.), streptokinase (also known as Streptase®, AstraZeneca, LP), pro-urokinase (Abbott Laboratories), urokinase (Abbott Laboratories), lanoteplase (Bristol-Myers Squibb Company), monteplase (Eisai Company, Ltd.), saruplase (also known as r-scu-PA and rescupase®, Grunenthal GmbH, Corp.), staphylokinase, and anisoylated plasminogen-streptokinase activator complex (also known as APSAC, Anistreplase and Eminase®, SmithKline Beecham Corp.). Thrombolytic agents also include other genetically engineered plasminogen activators.

Other possible agents include selective serotonin reuptake inhibitors, angiotensin receptor blockers, angiotensin-converting enzyme inhibitors, β-blockers and statins.

Antihypertensive agents can be used include those shown in Table 1. For example, useful antihypertensive agents can include, without limitation, an adrenergic blocker, a mixed alpha/beta adrenergic blocker, an alpha adrenergic blocker, a beta adrenergic blocker, an adrenergic stimulant, an angiotensin converting enzyme (ACE) inhibitor, an angiotensin II receptor antagonist, a calcium channel blocker, a diuretic, or a vasodilator. Additional hypertensive agents useful in the present invention are described in PCT Patent Application No. WO 99/11260, herein incorporated by reference.

TABLE 1

| Antihypertensive | | | |
|---|---|---|---|
| Classification | Compound Name | Typical Dosage | |
| adrenergic blocker | Phenoxybenzamine | 1-250 | mg/day |
| adrenergic blocker | Guanadrel | 5-60 | mg/day |
| adrenergic blocker | Guanethidine | | |
| adrenergic blocker | Reserpine | | |
| adrenergic blocker | Terazosin | 0.1-60 | mg/day |
| adrenergic blocker | Prazosin | 0.5-75 | mg/day |
| adrenergic blocker | Polythiazide | 0.25-10 | mg/day |
| adrenergic stimulant | Methyldopa | 100-4000 | mg/day |
| adrenergic stimulant | Methyldopate | 100-4000 | mg/day |
| adrenergic stimulant | Clonidine | 0.1-2.5 | mg/day |
| adrenergic stimulant | Chlorthalidone | 10-50 | mg/day |
| adrenergic blocker | Guanfacine | 0.25-5 | mg/day |
| adrenergic stimulant | Guanabenz | 2-40 | mg/day |
| adrenergic stimulant | Trimethaphan | | |
| alpha/beta adrenergic blocker | Carvedilol | 6-25 | mg bid |
| alpha/beta adrenergic blocker | Labetalol | 10-500 | mg/day |
| beta adrenergic blocker | Propranolol | 10-1000 | mg/day |
| beta adrenergic blocker | Metoprolol | 10-500 | mg/day |
| alpha adrenergic blocker | Doxazosin | 1-16 | mg/day |
| alpha adrenergic blocker | Phentolamine | | |
| angiotensin converting enzyme | Quinapril | 1-250 | mg/day |
| inhibitor | | | |
| angiotensin converting enzyme | perindopril erbumine | 1-25 | mg/day |
| inhibitor | | | |
| angiotensin converting enzyme | Ramipril | 0.25-20 | mg/day |
| inhibitor | | | |
| angiotensin converting enzyme | Captopril | 6-50 | mg bid or tid |
| inhibitor | | | |
| angiotensin converting enzyme | Trandolapril | 0.25-25 | mg/day |
| inhibitor | | | |
| angiotensin converting enzyme | Fosinopril | 2-80 | mg/day |
| inhibitor | | | |
| angiotensin converting enzyme | Lisinopril | 1-80 | mg/day |
| inhibitor | | | |
| angiotensin converting enzyme | Moexipril | 1-100 | mg/day |
| inhibitor | | | |
| angiotensin converting enzyme | Enalapril | 2.5040 | mg/day |
| inhibitor | | | |
| angiotensin converting enzyme | Benazepril | 10-80 | mg/day |
| inhibitor | | | |
| angiotensin II receptor | candesartan cilexetil | 2-32 | mg/day |
| antagonist | | | |
| angiotensin II receptor | Inbesartan | | |
| antagonist | | | |
| angiotensin II receptor | Losartan | 10-100 | mg/day |
| antagonist | | | |
| angiotensin II receptor | Valsartan | 20-600 | mg/day |
| antagonist | | | |
| calcium channel blocker | Verapamil | 100-600 | mg/day |
| calcium channel blocker | Diltiazem | 150-500 | mg/day |
| calcium channel blocker | Nifedipine | 1-200 | mg/day |
| calcium channel blocker | Nimodipine | 5-500 | mg/day |
| calcium channel blocker | Delodipine | | |
| calcium channel blocker | Nicardipine | 1-20 mg/hr i.v.; | |
| | | 5-100 mg/day oral | |
| calcium channel blocker | Isradipine | | |
| calcium channel blocker | Amlodipine | 2-10 | mg/day |
| diuretic | Hydrochlorothiazide | 5-100 | mg/day |
| diuretic | Chlorothiazide | 250-2000 | mg bid or tid |
| diuretic | Furosemide | 5-1000 | mg/day |
| diuretic | Bumetanide | | |
| diuretic | ethacrynic acid | 20-400 | mg/day |
| diuretic | Amiloride | 1-20 | mg/day |
| Diuretic | Triameterene | | |
| Diuretic | Spironolactone | 5-1000 | mg/day |
| Diuretic | Eplerenone | 10-150 | mg/day |
| Vasodilator | Hydralazine | 5-300 | mg/day |
| Vasodilator | Minoxidil | 1-100 | mg/day |
| Vasodilator | Diazoxide | 1-3 | mg/kg |
| Vasodilator | Nitroprusside | | |

Additional calcium channel blockers which are useful in the combinations of the present invention include, without limitation, those shown in Table 2.

TABLE 2

| Compound Name | Reference |
|---|---|
| bepridil | U.S. Pat. No. 3,962,238 or |
| | U.S. Reissue No. 30,577 |
| clentiazem | U.S. Pat. No. 4,567,175 |
| diltiazem | U.S. Pat. No. 3,562,257 |
| fendiline | U.S. Pat. No. 3,262,977 |
| gallopamil | U.S. Pat. No. 3,261,859 |
| mibefradil | U.S. Pat. No. 4,808,605 |
| prenylamine | U.S. Pat. No. 3,152,173 |
| semotiadil | U.S. Pat. No. 4,786,635 |
| terodiline | U.S. Pat. No. 3,371,014 |
| verapamil | U.S. Pat. No. 3,261,859 |
| aranipine | U.S. Pat. No. 4,572,909 |
| bamidipine | U.S. Pat. No. 4,220,649 |
| benidipine | European Patent Application |
| | Publication No. 106,275 |
| cilnidipine | U.S. Pat. No. 4,672,068 |
| efonidipine | U.S. Pat. No. 4,885,284 |
| elgodipine | U.S. Pat. No. 4,962,592 |
| felodipine | U.S. Pat. No. 4,264,611 |
| isradipine | U.S. Pat. No. 4,466,972 |
| lacidipine | U.S. Pat. No. 4,801,599 |
| lercanidipine | U.S. Pat. No. 4,705,797 |
| manidipine | U.S. Pat. No. 4,892,875 |
| nicardipine | U.S. Pat. No. 3,985,758 |
| nifendipine | U.S. Pat. No. 3,485,847 |
| nilvadipine | U.S. Pat. No. 4,338,322 |
| nimodipine | U.S. Pat. No. 3,799,934 |
| nisoldipine | U.S. Pat. No. 4,154,839 |
| nitrendipine | U.S. Pat. No. 3,799,934 |
| cinnarizine | U.S. Pat. No. 2,882,271 |
| flunarizine | U.S. Pat. No. 3,773,939 |
| lidoflazine | U.S. Pat. No. 3,267,104 |
| lomerizine | U.S. Pat. No. 4,663,325 |
| Bencyclane | Hungarian Patent No. 151,865 |
| Etafenone | German Patent No. 1,265,758 |
| Perhexiline | British Patent No. 1,025,578 |

Additional ACE inhibitors which are useful in the combinations of the present invention include, without limitation, those shown in Table 3.

TABLE 3

| Compound Name | Reference |
|---|---|
| alacepril | U.S. Pat. No. 4,248,883 |
| benazepril | U.S. Pat. No. 4,410,520 |
| captopril | U.S. Pat. Nos. 4,046,889 and |
| | 4,105,776 |
| ceronapril | U.S. Pat. No. 4,452,790 |
| delapril | U.S. Pat. No. 4,385,051 |
| enalapril | U.S. Pat. No. 4,374,829 |
| fosinopril | U.S. Pat. No. 4,337,201 |
| imadapril | U.S. Pat. No. 4,508,727 |
| lisinopril | U.S. Pat. No. 4,555,502 |
| moveltopril | Belgian Patent No. 893,553 |
| perindopril | U.S. Pat. No. 4,508,729 |
| quinapril | U.S. Pat. No. 4,344,949 |
| ramipril | U.S. Pat. No. 4,587,258 |
| Spirapril | U.S. Pat. No. 4,470,972 |
| Temocapril | U.S. Pat. No. 4,699,905 |
| Trandolapril | U.S. Pat. No. 4,933,361 |

Additional beta adrenergic blockers which are useful in the combinations of the present invention include, without limitation, those shown in Table 4.

TABLE 4

| Compound Name | Reference |
|---|---|
| acebutolol | U.S. Pat. No. 3,857,952 |
| alprenolol | Netherlands Patent Application No. |
| | 6,605,692 |
| amosulalol | U.S. Pat. No. 4,217,305 |
| arotinolol | U.S. Pat. No. 3,932,400 |
| atenolol | U.S. Pat. No. 3,663,607 or |
| | U.S. Pat. No. 3,836,671 |
| befunolol | U.S. Pat. No. 3,853,923 |
| betaxolol | U.S. Pat. No. 4,252,984 |
| bevantolol | U.S. Pat. No. 3,857,981 |
| bisoprolol | U.S. Pat. No. 4,171,370 |
| bopindolol | U.S. Pat. No. 4,340,641 |
| bucumolol | U.S. Pat. No. 3,663,570 |
| bufetolol | U.S. Pat. No. 3,723,476 |
| bufuralol | U.S. Pat. No. 3,929,836 |
| bunitrolol | U.S. Pat. Nos. 3,940,489 and |
| | U.S. Pat. No. 3,961,071 |
| buprandolol | U.S. Pat. No. 3,309,406 |
| butiridine hydrochloride | French Patent No. 1,390,056 |
| butofilolol | U.S. Pat. No. 4,252,825 |
| carazolol | German Patent No. 2,240,599 |
| carteolol | U.S. Pat. No. 3,910,924 |
| carvedilol | U.S. Pat. No. 4,503,067 |
| celiprolol | U.S. Pat. No. 4,034,009 |
| cetamolol | U.S. Pat. No. 4,059,622 |
| cloranolol | German Patent No. 2,213,044 |
| dilevalol | Clifton et al., Journal of Medicinal |
| | Chemistry, 1982 25, 670 |
| epanolol | European Patent Publication |
| | Application No. 41,491 |
| indenolol | U.S. Pat. No. 4,045,482 |
| labetalol | U.S. Pat. No. 4,012,444 |
| levobunolol | U.S. Pat. No. 4,463,176 |
| mepindolol | Seeman et al., Helv. Chim. Acta, |
| | 1971, 54, 241 |
| metipranolol | Czechoslovakian Patent Application |
| | No. 128,471 |
| metoprolol | U.S. Pat. No. 3,873,600 |
| moprolol | U.S. Pat. No. 3,501,769 |
| nadolol | U.S. Pat. No. 3,935,267 |
| nadoxolol | U.S. Pat. No. 3,819,702 |
| nebivalol | U.S. Pat. No. 4,654,362 |
| nipradilol | U.S. Pat. No. 4,394,382 |
| oxprenolol | British Patent No. 1,077,603 |
| perbutolol | U.S. Pat. No. 3,551,493 |
| pindolol | Swiss Patent Nos. 469,002 and |
| | Swiss Patent Nos. 472,404 |
| practolol | U.S. Pat. No. 3,408,387 |
| pronethalol | British Patent No. 909,357 |
| propranolol | U.S. Pat. Nos. 3,337,628 and |
| | U.S. Pat. Nos. 3,520,919 |
| sotalol | Uloth et al., Journal of Medicinal |
| | Chemistry, 1966, 9, 88 |
| sufinalol | German Patent No. 2,728,641 |
| talindol | U.S. Pat. Nos. 3,935,259 and |
| | U.S. Pat. Nos. 4,038,313 |
| tertatolol | U.S. Pat. No. 3,960,891 |
| tilisolol | U.S. Pat. No. 4,129,565 |
| timolol | U.S. Pat. No. 3,655,663 |
| toliprolol | U.S. Pat. No. 3,432,545 |
| Xibenolol | U.S. Pat. No. 4,018,824 |

Additional alpha adrenergic blockers which are useful in the combinations of the present invention include, without limitation, those shown in Table 5.

TABLE 5

| Compound Name | Reference |
|---|---|
| amosulalol | U.S. Pat. No. 4,217,307 |
| arotinolol | U.S. Pat. No. 3,932,400 |
| dapiprazole | U.S. Pat. No. 4,252,721 |
| doxazosin | U.S. Pat. No. 4,188,390 |
| fenspirlde | U.S. Pat. No. 3,399,192 |
| indoramin | U.S. Pat. No. 3,527,761 |
| labetalol | U.S. Pat. No. 4,012,444 |
| naftopidil | U.S. Pat. No. 3,997,666 |
| nicergoline | U.S. Pat. No. 3,228,943 |
| prazosin | U.S. Pat. No. 3,511,836 |
| tamsulosin | U.S. Pat. No. 4,703,063 |
| Tolazoline | U.S. Pat. No. 2,161,938 |
| Trimazosin | U.S. Pat. No. 3,669,968 |
| Yohimbine | Raymond-Hamet, J. Pharm. Chim., |
| | 19, 209 (1934) |

Additional angiotensin II receptor antagonists, which are useful in the combinations of the present invention include, without limitation, those shown in Table 6.

TABLE 6

| Compound Name | Reference |
|---|---|
| Candesartan | U.S. Pat. No. 5,196,444 |
| Eprosartan | U.S. Pat. No. 5,185,351 |
| Irbesartan | U.S. Pat. No. 5,270,317 |
| Losartan | U.S. Pat. No. 5,138,069 |
| Valsartan | U.S. Pat. No. 5,399,578 |

Additional vasodilators which are useful in the combinations of the present invention include, without limitation, those shown in Table 7.

TABLE 7

| Compound Name | Reference |
|---|---|
| aluminum nicotinate | U.S. Pat. No. 2,970,082 |
| amotriphene | U.S. Pat. No. 3,010,965 |
| bamethan | Corrigan et al., Journal of the |
| | American Chemical Society, 1945, |
| | 67, 1894 |
| bencyclane | Hungarian Patent No. 151,865 |
| bendazol | J. Chem. Soc., 1968, 2426 |
| benfurodil hemisuccinate | U.S. Pat. No. 3,355,463 |
| benziodarone | U.S. Pat. No. 3,012,042 |
| betahistine | Walter et al., Journal of the American |
| | Chemical Society, 1941, 63, 2771 |
| bradykinin | Hamburg et al., Arch. Biochem. |
| | Biophys., 1958, 76, 252 |
| brovincamine | U.S. Pat. No. 4,146,643 |
| bufeniode | U.S. Pat. No. 3,542,870 |
| buflomedil | U.S. Pat. No. 3,895,030 |
| butalamine | U.S. Pat. No. 3,338,899 |
| cetiedil | French Patent No. 1,460,571 |
| chloracizine | British Patent No. 740,932 |
| chromonar | U.S. Pat. No. 3,282,938 |
| ciclonicate | German Patent No. 1,910,481 |
| cinepazide | Belgian Patent No. 730,345 |
| cinnarizine | U.S. Pat. No. 2,882,271 |
| citicoline | Kennedy et al., Journal of the |
| | American Chemical Society, 1955, |
| | 77, 250 or synthesized as disclosed in |
| | Kennedy, Journal of Biological |
| | Chemistry, 1956, 222, 185 |
| clobenfural | British Patent No. 1,160,925 |
| clonitrate | see Annalen, 1870, 155, 165 |
| cloricromen | U.S. Pat. No. 4,452,811 |
| cyclandelate | U.S. Pat. No. 2,707,193 |
| diisopropylamine | Neutralization of dichloroacetic acid |
| dichloroacetate | with diisopropyl amine |
| diisopropylamine | British Patent No. 862,248 |
| dichloroacetate | |
| dilazep | U.S. Pat. No. 3,532,685 |
| dipyridamole | British Patent No. 807,826 |
| droprenilamine | German Patent No. 2,521,113 |
| ebumamonine | Hermann et al., Journal of the |
| | American Chemical Society, 1979, |
| | 101, 1540 |
| efloxate | British Patent Nos. 803,372 and |
| | 824,547 |
| eledoisin | British Patent No. 984,810 |
| erythrityl | May be prepared by nitration of |
| | erythritol according to methods well- |
| | known to those skilled in the art. See |
| | e.g., Merck Index. |
| etafenone | German Patent No. 1,265,758 |
| fasudil | U.S. Pat. No. 4,678,783 |
| fendiline | U.S. Pat. No. 3,262,977 |
| fenoxedil | U.S. Pat. No. 3,818,021 or German |
| | Patent No. 1,964,712 |
| floredil | German Patent No. 2,020,464 |
| flunarizine | German Patent No. 1,929,330 or |
| | French Patent No. 2,014,487 |
| flunarizine | U.S. Pat. No. 3,773,939 |
| ganglefene | U.S.S.R. Patent No. 115,905 |
| hepronicate | U.S. Pat. No. 3,384,642 |
| hexestrol | U.S. Pat. No. 2,357,985 |
| hexobendine | U.S. Pat. No. 3,267,103 |
| ibudilast | U.S. Pat. No. 3,850,941 |
| ifenprodil | U.S. Pat. No. 3,509,164 |
| iloprost | U.S. Pat. No. 4,692,464 |
| inositol | Badgett et al., Journal of the |
| | American Chemical Society, 1947, |
| | 69, 2907 |
| isoxsuprine | U.S. Pat. No. 3,056,836 |
| itramin tosylate | Swedish Patent No. 168,308 |
| kallidin | Biochem. Biophys. Re&Commun., |
| | 1961, 6, 210 |
| kallikrein | German Patent No. 1,102,973 |
| khellin | Baxter et al., Journal of the Chemical |
| | Society, 1949, S 30 |
| lidofiazine | U.S. Pat. No. 3,267,104 |
| lomerizine | U.S. Pat. No. 4,663,325 |
| mannitol hexanitrate | May be prepared by the nitration of |
| | mannitol according to methods well- |
| | known to those skilled in the art |
| medibazine | U.S. Pat. No. 3,119,826 |
| moxisylyte | German Patent No. 905,738 |
| nafronyl | U.S. Pat. No. 3,334,096 |
| nicametate | Blicke & Jenner, J. Am. Chem. Soc., |
| | 64, 1722 (1942) |
| nicergoline | U.S. Pat. No. 3,228,943 |
| nicofuranose | Swiss Patent No. 366,523 |
| nimodipine | U.S. Pat. No. 3,799,934 |
| nitroglycerin | Sobrero, Ann., 64, 398 (1847) |
| nylidrin | U.S. Pat. Nos. 2,661,372 and |
| | 2,661,373 |
| papaverine | Goldberg, Chem. Prod. Chem. News, |
| | 1954,17,371 |
| pentaerythritol tetranitrate | U.S. Pat. No. 2,370,437 |
| pentifylline | German Patent No. 860,217 |
| pentoxifylline | U.S. Pat. No. 3,422,107 |
| pentrinitrol | German Patent No. 638,422-3 |
| perhexilline | British Patent No. 1,025,578 |
| pimefylline | U.S. Pat. No. 3,350,400 |
| piribedil | U.S. Pat. No. 3,299,067 |
| prenylamine | U.S. Pat. No. 3,152,173 |
| propatyl nitrate | French Patent No. 1,103,113 |
| prostaglandin E1 | May be prepared by any of the |
| | methods referenced in the Merck |
| | Index, Twelfth Edition, Budaved, |
| | Ed., New Jersey, 1996, p. 1353 |
| suloctidil | German Patent No. 2,334,404 |
| tinofedrine | U.S. Pat. No. 3,563,997 |
| tolazoline | U.S. Pat. No. 2,161,938 |
| trapidil | East German Patent No. 55,956 |
| tricromyl | U.S. Pat. No. 2,769,015 |
| trimetazidine | U.S. Pat. No. 3,262,852 |
| trolnitrate phosphate | French Patent No. 984,523 or |
| | German Patent No. 830,955 |
| vincamine | U.S. Pat. No. 3,770,724 |
| vinpocetine | U.S. Pat. No. 4,035,750 |
| Viquidil | U.S. Pat. No. 2,500,444 |
| Visnadine | U.S. Pat. Nos. 2,816,118 and |
| | 2,980,699 |
| xanthinol niacinate | German Patent No. 1,102,750 or |
| | Korbonits et al., Acta. Pharm. Hung., |
| | 1968, 38, 98 |

Additional diuretics which are useful in the combinations of the present invention include, without limitation, those shown in Table 8.

TABLE 8

| Compound Name | Reference |
|---|---|
| Acetazolamide | U.S. Pat. No. 2,980,676 |
| Althiazide | British Patent No. 902,658 |
| Amanozine | Austrian Patent No. 168,063 |
| Ambuside | U.S. Pat. No. 3,188,329 |
| Amiloride | Belgian Patent No. 639,386 |
| Arbutin | Tschb&habln, Annalen, 1930, 479, |
| | 303 |
| Azosemide | U.S. Pat. No. 3,665,002 |
| Bendroflumethiazide | U.S. Pat. No. 3,265,573 |
| Benzthiazide | McManus et al., 136^{th} Am. Soc. |
| | Meeting (Atlantic City, September |
| | 1959). Abstract of Papers, pp 13-0 |
| benzylhydro-chlorothiazide | U.S. Pat. No. 3,108,097 |
| Bumetanide | U.S. Pat. No. 3,634,583 |
| Butazolamide | British Patent No. 769,757 |
| Buthiazide | British Patent Nos. 861,367 and |
| | 885,078 |
| Chloraminophenamide | U.S. Pat. Nos. 2,809,194, |
| | 2,965,655 and 2,965,656 |
| Chlorazanil | Austrian Patent No. 168,063 |
| Chlorothiazide | U.S. Pat. Nos. 2,809,194 and |
| | 2,937,169 |
| Chlorthalidone | U.S. Pat. No. 3,055,904 |
| Clofenamide | Olivier, Rec. Trav. Chim., 1918, 37, |
| | 307 |
| Clopamide | U.S. Pat. No. 3,459,756 |
| Clorexolone | U.S. Pat. No. 3,183,243 |
| Cyclopenthiazide | Belgian Patent No. 587,225 |
| Cyclothiazide | Whitehead et al., Journal of Organic |
| | Chemistry, 1961, 26, 2814 |
| Disulfamide | British Patent No. 851,287 |
| Epithiazide | U.S. Pat. No. 3,009,911 |
| ethacrynic acid | U.S. Pat. No. 3,255,241 |
| Ethiazide | British Patent No. 861,367 |
| Ethoxolamide | British Patent No. 795,174 |
| Etozolin | U.S. Pat. No. 3,072,653 |
| Fenquizone | U.S. Pat. No. 3,870,720 |
| Furosemide | U.S. Pat. No. 3,058,882 |
| Hydracarbazine | British Patent No. 856,409 |
| Hydrochlorothiazide | U.S. Pat. No. 3,164,588 |
| Hydroflumethiazide | U.S. Pat. No. 3,254,076 |
| Indapamide | U.S. Pat. No. 3,565,911 |
| Isosorbide | U.S. Pat. No. 3,160,641 |
| Mannitol | U.S. Pat. No. 2,642,462; or |
| | 2,749,371; or 2,759,024 |
| Mefruside | U.S. Pat. No. 3,356,692 |
| Methazolamide | U.S. Pat. No. 2,783,241 |
| Methyclothiazide | Close et al., Journal of the American |
| | Chemical Society, 1960, 82, 1132 |
| Meticrane | French Patent Nos. M2790 and |
| | 1,365,504 |
| Metochalcone | Freudenberg et al., Ber., 1957, 90, |
| | 957 |
| Metolazone | U.S. Pat. No. 3,360,518 |
| Muzolimine | U.S. Pat. No. 4,018,890 |
| Paraflutizide | Belgian Patent No. 620,829 |
| Perhexiline | British Patent No. 1,025,578 |
| Piretanide | U.S. Pat. No. 4,010,273 |
| Polythiazide | U.S. Pat. No. 3,009,911 |
| Quinethazone | U.S. Pat. No. 2,976,289 |
| Teclothiazide | Close et al., Journal of the American |
| | Chemical Society, 1960, 82, 1132 |
| Ticrynafen | U.S. Pat. No. 3,758,506 |
| Torasemide | U.S. Pat. No. 4,018,929 |
| Triamterene | U.S. Pat. No. 3,081,230 |
| Trichlormethiazide | deStevens et al., Experientia, 1960, |
| | 16, 113 |
| Tripamide | Japanese Patent No. 73 05,585 |
| Urea | Can be purchased from commercial |
| | sources |
| Xipamide | U.S. Pat. No. 3,567,777 |

### Pharmaceutical Compositions

The compounds optionally are administered in a pharmaceutical composition comprising a pharmaceutically acceptable carrier. The carrier(s) should be acceptable in the sense of being compatible with the other ingredients of the formulation and not deleterious to the recipient thereof.

The formulations include those suitable for oral, parenteral (including subcutaneous, intradermal, intramuscular, intravenous and intraarticular), rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. All methods include the step of bringing into association a compound or a pharmaceutically acceptable salt or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampuls and vials, and may be stored in a freeze-dried (lyophilized) condition requiring only the addition of the sterile liquid carrier, for example, saline, water-for-injection, immediately prior to use. Extemporaneous injection solutions and suspensions may be prepared from sterile powders, granules and tablets of the kind previously described.

Formulations for rectal administration may be presented as a suppository with the usual carriers such as cocoa butter or polyethylene glycol.,

Formulations for topical administration in the mouth, for example buccally or sublingually, include lozenges comprising the active ingredient in a flavored basis such as sucrose and acacia or tragacanth, and pastilles comprising the active ingredient in a basis such as gelatin and glycerin or sucrose and acacia.

Exemplary unit dosage formulations are those containing an effective dose, as herein below recited, or an appropriate fraction thereof, of the active ingredient. The dosage is selected in accordance with a variety of factors including type, species, age, weight, sex and medical condition of the individual; the severity of the condition; the route of administration; the renal and hepatic function of the individual; and the particular compound or salt or ester thereof to be employed. Consideration of these factors is well within the purview of the ordinary skilled clinician.

It should be understood that in addition to the ingredients particularly mentioned above, the formulations of this invention may include other agents conventional in the art having regard to the type of formulation in question, for example those suitable for oral administration may include flavoring agents.

The compounds may be administered orally or via injection at a dose, for example, of from 0.001 to 2500 mg/kg per day. The dose range for humans is generally from 0.005 mg to 10 g/day. Tablets or other forms of presentation provided in discrete units may conveniently contain an amount of a compound as disclosed herein which is effective at such dosage or as a multiple of the same, for instance, units containing 5 mg to 500 mg, usually around 10 mg to 200 mg.

The precise amount of compound administered to a patient will be adjusted as needed. However, the dose employed will depend on a number of factors, including the age and sex of the patient, the precise disorder being treated, and its severity. Also, the route of administration may vary depending on the condition and its severity.

The compounds can also be administered via a catheter or stent, for example, by use of an intraluminal stent. Although stents are commonly used as part of an angioplasty procedure, intraluminal stents can be used to maintain or control any bodily luminal opening. The compound could be used alone or as part of a composition allowing for a controlled release of the therapeutically active compound. The compounds could be coated on the stent or made a part of the stent. They may be layered so as to provide limited release of the active compound, or used in any manner known in the art. See U.S. Patent Application Nos. 20010029660 and 20010032014, herein incorporated by reference in their entirety.

### EXAMPLES

Platelet aggregation and platelet activity biomarkers were tested as follows:

Different doses of Compound A, below, were tested:

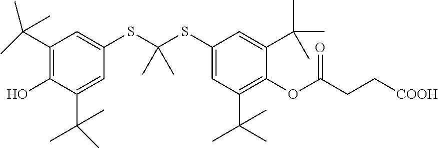

The effect of doses (15 μg/ml to 90 μg/ml) of Compound A on in vitro platelet characteristics in subjects with multiple factors for vascular disease was tested.

The in vitro effects of pre-incubation with escalating concentrations of Compound A on platelet aggregation, and expression of major surface receptors by flow cytometry, were assessed in 20 aspirin naïve volunteers with multiple risk factors for vascular disease.

Pretreatment of blood samples with 15-30-60-90 μg/ml Compound A resulted in the uniformed significant inhibition of platelet aggregation induced by ADP. Surface platelet expression of PECAM-1 (CD31), GP IIb/IIIa (CD41a) antigen, and activity with PAC-1 antibody, and GP Ib (CD42b) were decreased in the Compound A-pretreated samples. Formation of platelet-monocyte microparticles (CD14+CD151), and PAR-1 thrombin receptor expression of both intact, and active epitopes were also reduced.

For collagen-induced aggregation, thrombospondin (CD36), vitronectin receptor (CD51/CD61), P-selectin (CD62p), LAMP-3 (CD63), LAMP-1 (CD107a), CD40-ligand (CD 154), GP37 (CD165), there was little or no effect by Compound A in the assay.

Thus, Compound A was found to have a good anti-platelet profile, and inhibited platelets.

The results are shown in Table 9 below. Demographics are shown in Table 10.

TABLE 9
Effect of preincubation with Compound A on platelet activity
biomarkers in subjects with multiple risk factors for vascular disease.

| | Control | | | | |
|---|---|---|---|---|---|
| | +0.1% | Compound A | | | |
| Control | alcohol | 15 μg/mL | 30 μg/mL | 60 μg/mL | 90 μg/mL |

Conventional platelet aggregation, %

| | | | | | | |
|---|---|---|---|---|---|---|
| ADP 5 μM | 65.75 ± 6.4 | 68 ± 7.1 | 39.95 ± 11.9 | 40.4 ± 6.9 | 42.05 ± 7.2 | 42.65 ± 6.3 |
| Collagen 5 μg,/ml | 67.15 ± 7.1 | 68.2 ± 7.2 | 64.95 ± 5.1 | 65.6 ± 8.7 | 66.6 ± 6.8 | 65.5 ± 6.0 |

Flow cytometry

| | | | | | | |
|---|---|---|---|---|---|---|
| CD31 | 60.4 ± 5.4 | 60.7 ± 5.5 | 58.4 ± 5.7 | 57.8 ± 6.5 | 55.8 ± 7.1 | 50.9 ± 8.9 |
| CD41a | 183.5 ± 17.4 | 177.3 ± 14.6 | 185.3 ± 21.3 | 170.3 ± 16.7 | 165.5 ± 16.7 | 160.9 ± 23.2 |
| PAC-1 | 11.8 ± 1.1 | 12.0 ± 1.3 | 11.4 ± 1.1 | 11.2 ± 1.4 | 10.9 ± 2.2 | 10.3 ± 1.8 |
| CD42b | 156.4 ± 9.4 | 154.9 ± 10.1 | 151.6 ± 11.2 | 142.8 ± 11.4 | 137.7 ± 8.9 | 111.2 ± 11.2 |
| CD51/61 | 11.0 ± 2.1 | 11.0 ± 2.0 | 10.9 ± 2.1 | 11.4 ± 1.6 | 11.8 ± 2.1 | 10.2 ± 1.7 |
| CD62p | 9.6 ± 2.1 | 9.3 ± 1.7 | 9.8 ± 1.8 | 10.2 ± 1.9 | 10.5 ± 1.8 | 9.7 ± 1.8 |
| CD63 | 7.0 ± 1.7 | 7.4 ± 1.3 | 7.0 ± 1.4 | 7.4 ± 1.4 | 6.8 ± 1.5 | 6.7 ± 1.4 |
| CD107a | 6.0 ± 1.4 | 6.5 ± 1.5 | 6.7 ± 1.5 | 6.4 ± 1.4 | 6.1 ± 1.5 | 6.4 ± 1.6 |
| CD151 + 14 | 132.6 ± 11.0 | 129.0 ± 11.1 | 131.4 ± 11.8 | 107.6 ± 15.4 | 98.7 ± 12.9 | 97.9 ± 10.8 |
| CD154 | 6.5 ± 1.2 | 6.2 ± 1.1 | 6.6 ± 1.0 | 6.7 ± 0.9 | 6.8 ± 1.1 | 7.0 ± 1.1 |
| CD165 | 25.7 ± 3.2 | 26.1 ± 3.1 | 26.9 ± 2.7 | 24.7 ± 2.7 | 26.4 ± 3.7 | 25.5 ± 3.2 |
| PAR-1 | 42.8 ± 6.2 | 43.34.8 | 40.6 ± 5.5 | 36.1 ± 4.7 | 37.7 ± 5.5 | 34.3 ± 5.2 |
| (WEDE15) | | | | | | |
| PAR-1 | 27.6 ± 3.9 | 27.0 ± 3.7 | 25.0 ± 4.2 | 23.9 ± 3.8 | 24.4 ± 2.8 | 19.4 ± 3.5 |
| (SPAN-12) | | | | | | |
| Thrombospondin | 10.6 ± 1.6 | 10.0 ± 2.3 | 10.1 ± 1.9 | 11.0 ± 2.3 | 10.3 ± 1.9 | 9.6 ± 2.0 |

TABLE 10
Demographics, and Clinical Characteristics

| Parameter | | |
|---|---|---|
| Demographics | | |
| Age | 32.2 ± 5.0 | |
| Male | 13 | (65%) |
| Caucasians | 13 | (65%) |
| African-American | 7 | (35%) |
| Multiple risk factors | | |
| Obesity | 6 | (30%) |
| Family history | 10 | (50%) |
| Hyperlipidemia | 4 | (20%) |
| Smoking (present or history of past smoking) | 13 | (65%) |
| Sedentary life style | 14 | (70%) |

Those skilled in the art will recognize, or be able to ascertain using no more than routine experimentation, many equivalents to the specific embodiments of the invention described herein. Such equivalents are intended to be encompassed by the following claims.

1. A method of reducing a platelet activation state of an individual in need thereof, the method comprising administering an effective amount of a compound of formula: 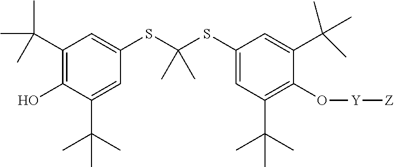
or a pharmaceutically acceptable salt wherein:
Y is a bond;
Z is selected from the group consisting of hydroxyC₁₋₆alkyl, C₁₋₆alkoxyC₁₋₆alkyl, and carboxyC₁-₆alkyl, wherein all may optionally be substituted by one or more R₅;
R₅ is independently selected from the group selected from hydroxy, amino, halo, COOH, COOR₇, CH(OH)R₇, NHR₇, NR₇R₇, C(O)NH₂, C(O)NHR₇, CONR₇R₇, OSO₃H, SO₃H, SO₂NHR₇, SO₂NR₇R₇, P(O)(OH)OR₇, P(O)(OH)R₇, P(O)HR₇, P(OR₇)₂, P(O)R₇(OR₇), OPO₃H, PO₃H₂, and hydroxymethyl, wherein when possible, all may be optionally substituted by one or more R₆;
R₆ is independently selected from the group consisting of hydroxy, C₁₋₁₀alkyl, C₁₋₁₀alkoxy, acyloxy, halo, nitro, amino, cyano, haloC₁₋₁₀alkyl, C₁₋₁₀alkylamino, diC₁₋₁₀alkylamino, acyl, and acyloxy;
R₇ is independently selected from the group consisting of C₁₋₆alkyl, C₂₋₁₀alkenyl, C₂₋₆alkynyl, C₁₋₆alkoxy, C₁₋₆alkoxycarbonylC₁₋₆alkyl, carboxyC₁₋₆alkyl, and C₁₋₆alkylcarboxyC₁₋₆alkyl, wherein all may be optionally substituted by one or more R₈; and
R₈ is independently selected from the group consisting of hydroxy, C₁₋₆alkyl, C₁₋₆alkoxy, acyloxy, halo, amino, cyano, and carboxy.

2. The method of claim 1, wherein:
Z is carboxyC₁₋₆alkyl, optionally substituted by one or more R₅;
R₅ is independently selected from the group consisting of halo, COOH, COOR₇, CONH₂, CONHR₇, CONR₇R₇, and amino;
R₇ is independently selected from the group consisting of C₁₋₆alkyl, carboxyC₁₋₆alkyl, C₁₋₆alkoxycarbonylC₁₋₆alkyl, and C₁₋₆alkylcarboxyC₁₋₆alkyl, wherein all may be optionally substituted by one or more R₈; and
R₈ is independently selected from the group consisting of hydroxy, halo, amino, and carboxy.

3. The method of claim 2, wherein:
Z is carboxyC₁₋₆alkyl, optionally substituted by one or more R₅; and
R₅ is COOH.

4. The method of claim 3, wherein the compound is selected from the group consisting of:

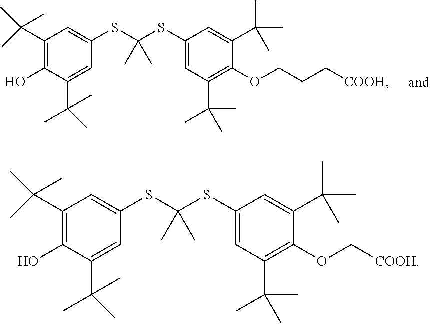

5. A method of reducing a platelet activation state of an individual in need thereof, the method comprising administering an effective amount of a compound of formula: 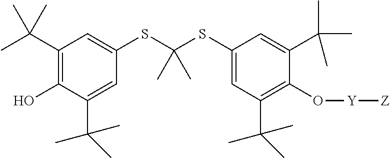
or a pharmaceutically acceptable salt wherein:
Y is 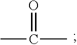
Z is selected from the group consisting of C₁₋₁₀alkyl, C₂₋₁₀alkenyl, C₂₋₁₀alkynyl, hydroxyC₁₋₁₀alkyl, aryl, heteroaryl, C₁₋₁₀alkaryl, arylC₁₋₁₀alkyl, heteroarylC₁₋₁₀alkyl, C₁₋₁₀alkoxyC₁₋₁₀alkyl, C₁₋₁₀alkylaminoC₁₋₁₀alkyl, carboxyC₁₋₁₀alkyl, C₁₋₁₀dialkylaminoC₁₋₁₀alkyl, aminoC₁₋₁₀alkyl, heterocycle, heterocyclC₁₋₁₀alkyl, R₇NH, R₇R₇N, carboxy, carbohydrate group, carbohydrate lactone group, and an alditol group wherein all may optionally be substituted by one or more R₅;
R₅ is independently selected from the group selected from hydroxy, C₁₋₁₀alkyl, C₁₋₁₀alkoxy, halo, nitro, amino, cyano, C₁₋₁₀alkylamino, diC₁₋₁₀alkylamino, acyl, acyloxy, COOH, COOR₇, OC(O)R₇, CH(OH)R₇, NHR₇, NR₇R₇, C(O)NH₂, C(O)NHR₇, CONR₇R₇, NHC(O)O—R₇, OSO₃H, SO₃H, SO₂NHR₇, SO₂NR₇R₇, P(O)(OH)OR₇, PO₂H₂ P(O)(OH)R₇, P(O)(OR₇)₂, P(O)R₇(OR₇), OPO₃H, PO₃H₂, hydroxymethyl, and cyclic phosphate, wherein when possible, all may be optionally substituted by one or more R₆;
R₆ is independently selected from the group consisting of hydroxy, C₁₋₁₀alkyl, C₁₋₁₀alkoxy, acyloxy, halo, nitro, amino, cyano, haloC₁₋₁₀alkyl, C₁₋₁₀alkylamino, diC₁₋₁₀alkylamino, acyl, and acyloxy;
R₇ is independently selected from the group consisting of C₁₋₁₀alkyl, C₂₋₁₀alkenyl, C₂₋₁₀alkynyl, C₁₋₁₀alkoxy, C₁₋₁₀alkoxycarbonylC₁₋₁₀alkyl, aryl, carboxyC₁₋₁₀alkyl, C₁₋₁₀alkylcarboxyC₁₋₁₀alkyl, C₁₋₁₀alkylcarboxyC₁₋₁₀aryl, heterocycle, heterocyclC₁₋₁₀alkyl, and heteroaryl, wherein all may be optionally substituted by one or more R₈; and
R₈ is independently selected from the group consisting of hydroxy, C₁₋₁₀alkyl, C₁₋₁₀alkoxy, acyloxy, halo, nitro, amino, cyano, and carboxy;
wherein two R₇ groups may come together to form a 4 to 7 membered ring.

6. The method of claim 5, wherein:
Z is selected from the group consisting of C₁₋₆alkyl, hydroxyC₁₋₆alkyl, C₁₋₆alkoxyC₁₋₆alkyl, and carboxyC₁₋₆alkyl, wherein all may optionally be substituted by one or more R₅;
R₅ is independently selected from the group selected from hydroxy, amino, halo, COOH, COOR₇, CH(OH)R₇, NHR₇, NR₇R₇, C(O)NH₂, C(O)NHR₇, CONR₇R₇, OSO₃H, SO₃H, SO₂NHR₇, SO₂NR₇R₇, P(O)(OH)OR₇, P(O)(OH)R₇, P(O)HR₇, P(OR₇)₂, P(O)R₇(OR₇), OPO₃H, PO₃H₂, and hydroxymethyl, wherein when possible, all may be optionally substituted by one or more R₆;
R₇ is independently selected from the group consisting of C₁₋₆alkyl, C₂₋₁₀alkenyl, C₂₋₆alkynyl, C₁₋₆alkoxy, C₁₋₆alkoxycarbonylC₁₋₆alkyl, carboxyC₁₋₆alkyl, and C₁₋₆alkylcarboxyC₁₋₆alkyl, wherein all may be optionally substituted by one or more R₈; and
R₈ is independently selected from the group consisting of hydroxy, C₁₋₆alkyl, C₁₋₆alkoxy, acyloxy, halo, amino, cyano, and carboxy.

7. The method of claim 6, wherein:
Z is C₁₋₆alkyl, optionally substituted by one or more R₅;
R₅ is independently selected from the group consisting of halo, COOH, COOR₇, CONH₂, CONHR₇, CONR₇R₇, and amino;
R₇ is independently selected from the group consisting of C₁₋₆alkyl, carboxyC₁₋₆alkyl, and C₁₋₆alkylcarboxyC₁₋₆alkyl, wherein all may be optionally substituted by one or more R₈; and
R₈ is independently selected from the group consisting of hydroxy, halo, amino, and carboxy.

8. The method of claim 7, wherein:
Z is C₁₋₆alkyl, optionally substituted by one or more R₅; and
R₅ is COOH.

9. The method of claim 8, wherein the compound is one of the following compounds or a pharmaceutically acceptable salt thereof:

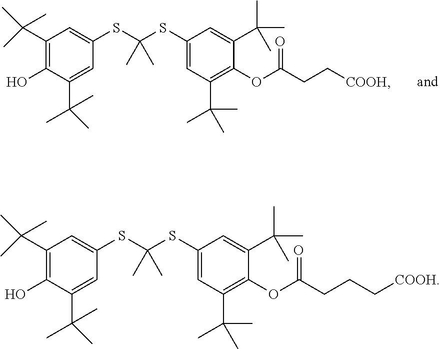

10. A method of reducing a platelet activation state of an individual in need thereof, the method comprising administering an effective amount of a compound, or a pharmaceutically acceptable salt thereof, wherein the compound is selected from the group consisting of:

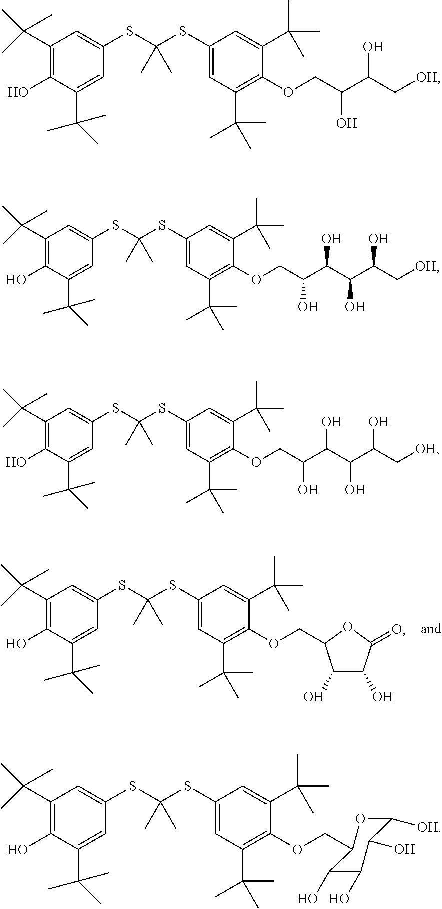

11. The method of claim 1, wherein the compound reduces the activity of protease activating receptors 1 or 4 (PAR-1 or PAR-4) in platelets of the individual.

12. The method of claim 11, further comprising comparing the expression level of platelet PAR-1/PAR-4 thrombin receptor expression from a sample taken from the individual after administration of the compound to a control, and detecting a decrease in the expression.

13. The method of claim 1, comprising comparing the level of at least one platelet activation marker from a sample taken from the individual, to a control, wherein the level is decreased after administration of the compound.

14. The method of claim 13, wherein the level of the platelet activation marker is reduced by at least about 10%.

15. The method of claim 1, wherein the compound is administered orally, intravenously, intramuscularly, subcutaneously, parenterally, nasally, by inhalation, by implant, or by suppository.

16. The method of claim 15, wherein the compound is administered orally in an amount between about 0.5 mg-2500 mg/daily.

17. A method of treating a vascular event, disease or disorder, that is a thrombotic or thromboembolic event, present in an individual, or which the individual is at risk of being afflicted with, the method comprising administering to the individual an effective amount of a compound of formula: 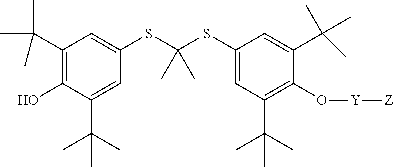
or a pharmaceutically acceptable salt thereof wherein:
Y is a bond;
Z is selected from the group consisting of hydroxyC₁₋₆alkyl, C₁₋₆alkoxyC₁₋₆alkyl, and carboxyC₁-₆alkyl, wherein all may optionally be substituted by one or more R₅;
R₅ is independently selected from the group selected from hydroxy, amino, halo, COOH, COOR₇, CH(OH)R₇, NHR₇, NR₇R₇, C(O)NH₂, C(O)NHR₇, CONR₇R₇, OSO₃H, SO₃H, SO₂NHR₇, SO₂NR₇R₇, P(O)(OH)OR₇, P(O)(OH)R₇, P(O)HR₇, P(OR₇)₂, P(O)R₇(OR₇), OPO₃H, PO₃H₂, and hydroxymethyl, wherein when possible, all may be optionally substituted by one or more R₆;
R₆ is independently selected from the group consisting of hydroxy, C₁₋₁₀alkyl, C₁₋₁₀alkoxy, acyloxy, halo, nitro, amino, cyano, haloC₁₋₁₀alkyl, C₁₋₁₀alkylamino, diC₁₋₁₀alkylamino, acyl, and acyloxy;
R₇ is independently selected from the group consisting of C₁₋₆alkyl, C₂₋₁₀alkenyl, C₂₋₆alkynyl, C₁₋₆alkoxy, C₁₋₆alkoxycarbonylC₁₋₆alkyl, carboxyC₁₋₆alkyl, and C₁₋₆alkylcarboxyC₁₋₆alkyl, wherein all may be optionally substituted by one or more R₈; and
R₈ is independently selected from the group consisting of hydroxy, C₁₋₆alkyl, C₁₋₆alkoxy, acyloxy, halo, amino, cyano, and carboxy.

18. The method of claim 17, wherein:
Z is carboxyC₁₋₆alkyl, optionally substituted by one or more R₅;
R₅ is independently selected from the group consisting of halo, COOH, COOR₇, CONH₂, CONHR₇, CONR₇R₇, and amino;
R₇ is independently selected from the group consisting of C₁₋₆alkyl, carboxyC₁₋₆-alkyl, C₁₋₆alkoxycarbonylC₁₋₆alkyl, and C₁₋₆alkylcarboxyC₁₋₆alkyl, wherein all may be optionally substituted by one or more R₈; and
R₈ is independently selected from the group consisting of hydroxy, halo, amino, and carboxy.

19. The method of claim 18, wherein:
Z is carboxyC₁₋₆alkyl, optionally substituted by one or more R₅; and R₅ is COOH.

20. The method of claim 19, wherein the compound is selected from the group consisting of:

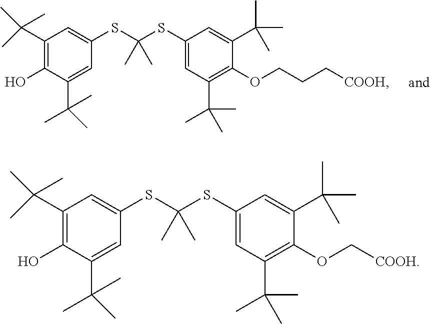

21. A method of treating a vascular event, disease or disorder, that is a thrombotic or thromboembolic event, that is present in an individual, or which the individual is at risk of being afflicted with, the method comprising administering to the individual an effective amount of a compound of formula: 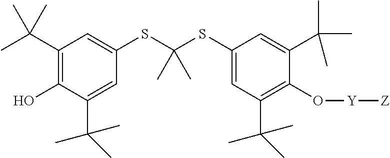
or a pharmaceutically acceptable salt thereof wherein:
Y is 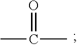
Z is selected from the group consisting of C₁₋₁₀alkyl, C₂₋₁₀alkenyl, C₂₋₁₀alkynyl, hydroxyC₁₋₁₀alkyl, aryl, heteroaryl, C₁₋₁₀alkaryl, arylC₁₋₁₀alkyl, heterarylC₁₋₁₀alkyl, C₁-₁₀alkoxyC₁₋₁₀alkyl, C₁₋₁₀alkylaminoC₁₋₁₀alkyl, carboxyC₁₋₁₀alkyl, C₁₋₁₀dialkylaminoC₁₋₁₀alkyl, aminoC₁₋₁₀alkyl, heterocycle, heterocyclC₁₋₁₀alkyl, R₇NH, R₇R₇N, carboxy, carbohydrate group, carbohydrate lactone group, and an alditol group wherein all may optionally be substituted by one or more R₅;
R₅ is independently selected from the group selected from hydroxy, C₁₋₁₀alkyl, C₁₋₁₀alkoxy, halo, nitro, amino, cyano, C₁₋₁₀alkylamino, diC₁₋₁₀alkylamino, acyl, acyloxy, COOH, COOR₇, OC(O)R₇, CH(OH)R₇, NHR₇, NR₇R₇, C(O)NH₂, C(O)NHR₇, CONR₇R₇, NHC(O)O—R₇, OSO₃H, SO₃H, SO₂NHR₇, SO₂NR₇R₇, P(O)(OH)OR₇, PO₂H₂ P(O)(OH)R₇, P(O)(OR₇)₂, P(O)R₇(OR₇), OPO₃H, PO₃H₂, hydroxymethyl, and cyclic phosphate, wherein when possible, all may be optionally substituted by one or more R₆;
R₆ is independently selected from the group consisting of hydroxy, C₁₋₁₀alkyl, C₁₋₁₀alkoxy, acyloxy, halo, nitro, amino, cyano, haloC₁₋₁₀alkyl, C₁₋₁₀alkylamino, diC₁₋₁₀alkylamino, acyl, and acyloxy;
R₇ is independently selected from the group consisting of C₁₋₁₀alkyl, C₂₋₁₀alkenyl, C₂₋₁₀alkynyl, C₁₋₁₀alkoxy, C₁₋₁₀alkoxycarbonylC₁₋₁₀alkyl, aryl, carboxyC₁₋₁₀alkyl, C₁₋₁₀alkylcarboxyC₁₋₁₀alkyl, C₁₋₁₀alkylcarboxyC₁₋₁₀aryl, heterocycle, heterocyclC₁₋₁₀alkyl, and heteroaryl, wherein all may be optionally substituted by one or more R₈; and
R₈ is independently selected from the group consisting of hydroxy, C₁₋₁₀alkyl, C₁₋₁₀alkoxy, acyloxy, halo, nitro, amino, cyano, and carboxy;
wherein two R₇ groups may come together to form a 4 to 7 membered ring.

22. The method of claim 21, wherein:
Z is selected from the group consisting of C₁₋₆alkyl, hydroxyC₁₋₆alkyl, C₁₋₆alkoxyC₁-₆alkyl, and carboxyC₁₋₆alkyl, wherein all may optionally be substituted by one or more R₅;
R₅ is independently selected from the group selected from hydroxy, amino, halo, COOH, COOR₇, CH(OH)R₇, NHR₇, NR₇R₇, C(O)NH₂, C(O)NHR₇, CONR₇R₇, OSO₃H, SO₃H, SO₂NHR₇, SO₂NR₇R₇, P(O)(OH)OR₇, P(O)(OH)R₇, P(O)HR₇, P(OR₇)₂, P(O)R₇(OR₇), OPO₃H, PO₃H₂, and hydroxymethyl, wherein when possible, all may be optionally substituted by one or more R₆;
R₇ is independently selected from the group consisting of C₁₋₆alkyl, C₂₋₁₀alkenyl, C₂₋₆alkynyl, C₁₋₆alkoxy, C₁₋₆alkoxycarbonylC₁₋₆alkyl, carboxyC₁₋₆alkyl, and C₁₋₆alkylcarboxyC₁₋₆alkyl, wherein all may be optionally substituted by one or more R₈; and
R₈ is independently selected from the group consisting of hydroxy, C₁₋₆alkyl, C₁₋₆alkoxy, acyloxy, halo, amino, cyano, and carboxy.

23. The method of claim 22, wherein:
Z is C₁₋₆alkyl, optionally substituted by one or more R₅;
R₅ is independently selected from the group consisting of halo, COOH, COOR₇, CONH₂, CONHR₇, CONR₇R₇, and amino;
R₇ is independently selected from the group consisting of C₁₋₆alkyl, carboxyC₁₋₆alkyl, and C₁₋₆alkylcarboxyC₁₋₆alkyl, wherein all may be optionally substituted by one or more R₈; and
R₈ is independently selected from the group consisting of hydroxy, halo, amino, and carboxy.

24. The method of claim 23, wherein:
Z is C₁₋₆alkyl, optionally substituted by one or more R₅; and
R₅ is COOH.

25. The method of claim 24, wherein the compound is selected from the group consisting of

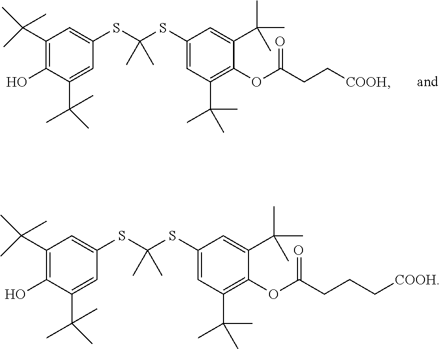

26. A method of treating a vascular event, disease or disorder, that is a thrombotic or thromboembolic event, that is present in an individual, or which the individual is at risk of being afflicted with, the method comprising administering to the individual an effective amount of a compound, or a pharmaceutically acceptable salt thereof, having a structure selected from the group consisting of:

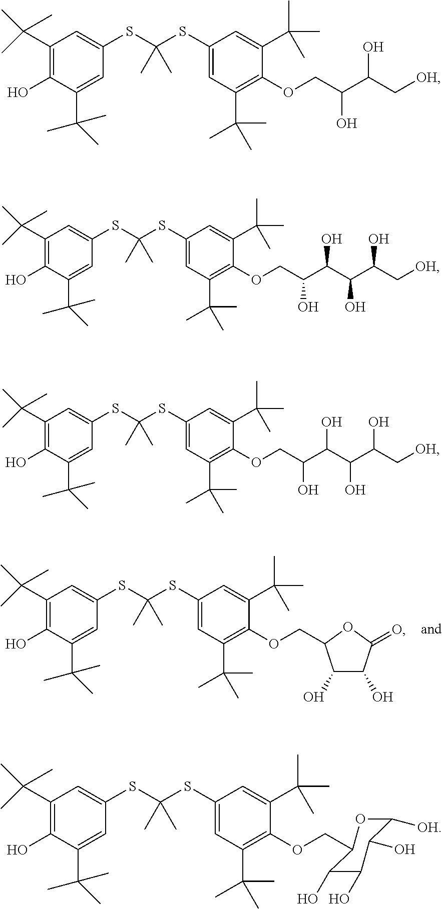

27. The method of claim 17, wherein the vascular event, disease or disorder is selected from a group consisting of: myocardial infarction, thrombosis, angina, stroke, pulmonary embolism, transient ischemic attack, deep vein thrombosis, atrial fibrillation, orthopedic surgery, thrombotic re-occlusion subsequent to a coronary intervention procedure, heart surgery or vascular surgery, peripheral vascular thrombosis, Syndrome X, heart failure, and a disorder in which a narrowing of at least one coronary artery occurs.

28. The method of claim 17, wherein the vascular event is a thromboembolic event.

29. The method of claim 17, wherein the compound reduces the activity of protease activating receptors 1 or 4 (PAR-1 or PAR-4) in platelets of the individual.

30. The method of claim 29, further comprising comparing the expression level of platelet PAR-1/PAR-4 thrombin receptor expression from a sample taken from the individual after administration of the compound to a control, and detecting a decrease in the expression.

31. The method of claim 17, the method comprising comparing the level of at least one platelet activation marker from a sample taken from the individual, to a control, wherein the level is decreased after administration of the compound.

32. The method of claim 31, wherein the level of the platelet activation marker is reduced by at least about 10%.

33. The method of claim 17, wherein the compound is administered orally, intravenously, intramuscularly, subcutaneously, parenterally, nasally, by inhalation, by implant, or by suppository.

34. The method of claim 33, wherein the compound is administered orally in an amount of about 0.5 mg to 2500 mg/daily.

35. The method of claim 17, wherein the method further comprises administering a second compound which is an anti-platelet compound, an anticoagulant or a thrombolyic agent.

36. The method of claim 1, wherein the individual is a human.
